SEQUENCE LISTING (32 sequences)

SEQ ID NO: 1 - protein, 5 aa, synthetic construct
  VARIANT 4: note = V or M
NYWXN

SEQ ID NO: 2 - protein, 17 aa, synthetic construct
  VARIANT 2: note = V or I
  VARIANT 9: note = T or S
  VARIANT 14: note = Q, R or K
  VARIANT 16: note = K or R
MXHPSGSEXRLNQXFXD

SEQ ID NO: 3 - protein, 9 aa, synthetic construct
VWSGNAFDY

SEQ ID NO: 4 - protein, 17 aa, synthetic construct
  VARIANT 1: note = L or M
  VARIANT 8: note = N or A
  VARIANT 9: note = S, A, or E
  VARIANT 13: note = R or K
XSSQSLLXXGNQXNYLT

SEQ ID NO: 5 - protein, 7 aa, synthetic construct
WASTRDS

SEQ ID NO: 6 - protein, 9 aa, synthetic construct
QNGFSFPFT

SEQ ID NO: 7 - protein, 118 aa, synthetic construct
QVQLQQPGAELVRPGTSVKLSCKASGYSFTNYWVNWVKQRPGQGLEWIGMVHPSGSETRLNQQFKDKATLTVDKSSNTAYMQLNRPTSEDSAVYYCARVWSGNAFDYWGQGTTLIVSS

SEQ ID NO: 8 - protein, 113 aa, synthetic construct
  VARIANT 31: note = N or A
  VARIANT 32: note = S, A or E
DIVMTQSPSSLTVTAGEKVTLNCLSSQSLLXXGNQRNYLTWYQQKPGQPPKLLIYWASTRDSGVPDRFTGGGSGVDFTLTIYRVQAEDLAFYYCQNGFSFPFTFGSGTKLEMK

SEQ ID NO: 9 - protein, 118 aa, synthetic construct
  VARIANT 28: note = S or T
  VARIANT 48: note = I or M
  VARIANT 74: note = K or T
  VARIANT 79: note = A or V
QVQLVQSGAEVKKPGASVKVSCKASGYXFTNYWVNWVRQAPGQGLEWXGMVHPSGSETRLNQQFKDKATLTVDXSTSTXYMELSSLRSEDTAVYYCARVWSGNAFDYWGQGTLVTVSS

SEQ ID NO: 10 - protein, 118 aa, synthetic construct
  VARIANT 67: note = R or K
  VARIANT 68: note = A or V
  VARIANT 70: note = L or M
  VARIANT 72: note = V or R
QVQLVQSGAEVKKPGASVKVSCKASGYSFTNYWVNWVRQAPGQGLEWIGMVHPSGSETRLNQQFKDXXTXTXDTSTSTVYMELSSLRSEDTAVYYCARVWSGNAFDYWGQGTLVTVSS

SEQ ID NO: 11 - protein, 113 aa, synthetic construct
  VARIANT 69: note = T or S
  VARIANT 75: note = V or T
  VARIANT 84: note = V or L
DIVMTQSPDSLAVSLGERATINCLSSQSLLASGNQRNYLTWYQQKPGQPPKLLIYWASTRDSGVPDRFXGSGSGXDFTLTISSXQAEDVAVYYCQNGFSFPFTFGQGTKLEIK

SEQ ID NO: 12 - protein, 118 aa, synthetic construct
QVQLQQPGAELVRPGTSVKLSCKASGYSFTNYWVNWVKQRPGQGLEWIGMIHPSGSETRLNQRFRDKTTLTVDRSSNTAYMQLNRPTSEDSAVYYCARVWSGNAFDYWGQGTTLIVSS

SEQ ID NO: 13 - protein, 113 aa, synthetic construct
DIVMTQSPSSLTVTAGEKVTLNCMSSQSLLNSGNQKNYLTWYQQKPGQPPKLLIYWASTRDSGVPDRFTGGGSGVDFTLTIYRVQAEDLAFYFCQNGFSFPFTFGSGTKLEMK

SEQ ID NO: 14 - protein, 118 aa, synthetic construct
QVQLQQPGAELVRPGTSVKLSCKASGYSFTNYWMNWVKQRPGQGLEWIGMIHPSGSESRLNQKFKDKATLTVDKSSNTAYMQLNRPTSADSAVYYCARVWSGNAFDYWGQGTTLIVSS

SEQ ID NO: 15 - protein, 113 aa, synthetic construct
DIVMTQSPSSLTVTAGEKVTLSCMSSQSLLNSGNQKNYLTWYQQKPGQPPKLLIFWASTRDSGVPDRFTGGGSGTLFTLTIYRVQAEDLAFYYCQNGFSFPFTFGSGTKLEMR

SEQ ID NO: 16 - protein, 330 aa, synthetic construct [sequence omitted]

SEQ ID NO: 17 - protein, 107 aa, synthetic construct [sequence omitted]

SEQ ID NO: 18 - protein, 448 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 220 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 602 aa, synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKHIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTELEHHHHHH

SEQ ID NO: 21 - DNA, 354 bp, synthetic construct
  source 1..354: mol_type = other DNA
caggtccagctgcagcagcctggggctgagctggtgaggcctggaacttcagtgaagctgtcctgcaaggcttctggttattccttcaccaactactgggtgaactgggtgaagcagaggcctggacaaggccttgagtggattggcatggttcatccttccggtagcgaaactaggttaaatcagcagttcaaggacaaggccacattgactgtagacaaatcgtccaatacagcctatatgcaactcaataggccgacatctgaagactctgcggtctattactgtgcaagggtctggtccggtaacgcctttgactactggggccaaggcaccactctcatagtctcctca

SEQ ID NO: 22 - DNA, 339 bp, synthetic construct
  source 1..339: mol_type = other DNA
gacattgtgatgacgcagtctccatcctccctgactgtgacagcaggagagaaggtcactttgaactgcctgtccagtcagagtctgttaaacagtggaaatcaaagaaactacttgacctggtaccagcagaaaccagggcagcctcctaaattgttgatctattgggcatccactagggattctggggtccctgatcgcttcacaggcggtggatctggagtagatttcactctcaccatctacagggtgcaggctgaagacctggcattttattactgtcagaatggtttcagttttccattcacgttcggctcggggacaaagttggaaatgaaa

SEQ ID NO: 23 - DNA, 354 bp, synthetic construct
  source 1..354: mol_type = other DNA
caagtgcagctgcagcagcctggcgccgagctggtgagacctggcacaagcgtgaagctgagctgcaaggctagcggctacagcttcaccaactactgggtgaactgggtgaagcagagacctggccaaggcctggagtggatcggcatggtgcaccctagcggcagcgagacaagactgaatcagcagttcaaggacaaggccaccctgaccgtggacaagagcagcaacaccgcctacatgcagctgaacagacctacaagcgaggacagcgccgtgtactactgcgctagagtgtggagcggcaacgccttcgactactggggccaaggcaccaccctgatcgtgagcagc

SEQ ID NO: 24 - DNA, 339 bp, synthetic construct
  source 1..339: mol_type = other DNA
gacatcgtgatgacacagagccctagcagcctgaccgtgaccgccggcgagaaggtgaccctgaactgcctgagcagccaaagcctgctgrmcrvbggcaatcagagaaactacctgacctggtatcagcagaagcctggacagcctcctaagctgctgatctactgggctagcacaagagacagcggcgtgcctgacagattcaccggcggaggcagcggcgtggacttcaccctgaccatctacagagtgcaagccgaggacctggccttctactactgtcagaacggcttcagcttccctttcaccttcggcagcggcaccaagctggagatgaag

SEQ ID NO: 25 - DNA, 354 bp, synthetic construct
  source 1..354: mol_type = other DNA
caagtgcagctggtgcagagcggcgccgaggtgaagaagcctggcgctagcgtgaaggtgagctgcaaggctagcggctacascttcaccaactactgggtgaactgggtgagacaagcccctggccaaggcctggagtggatcggcatggtgcaccctagcggcagcgagacaagactgaatcagcagttcaaggacaaggccaccctgaccgtggacamragcacaagcaccgystacatggagctgagcagcctgagaagcgaggacaccgccgtgtactactgcgctagagtgtggagcggcaacgccttcgactactggggccaaggcaccctggtgaccgtgagcagc

SEQ ID NO: 26 - DNA, 354 bp, synthetic construct
  source 1..354: mol_type = other DNA
caagtgcagctggtgcagagcggcgccgaggtgaagaagcctggcgctagcgtgaaggtgagctgcaaggctagcggctacagcttcaccaactactgggtgaactgggtgagacaagcccctggccaaggcctggagtggatsggcatggtgcaccctagcggcagcgagacaagactgaatcagcagttcaaggacarrgysaccctgaccgtggacacaagcacaagcaccgtgtacatggagctgagcagcctgagaagcgaggacaccgccgtgtactactgcgctagagtgtggagcggcaacgccttcgactactggggccaaggcaccctggtgaccgtgagcagc

SEQ ID NO: 27 - DNA, 354 bp, synthetic construct
  source 1..354: mol_type = other DNA
caagtgcagctggtgcagagcggcgccgaggtgaagaagcctggcgctagcgtgaaggtgagctgcaaggctagcggctacagcttcaccaactactgggtgaactgggtgagacaagcccctggccaaggcctggagtggatcggcatggtgcaccctagcggcagcgagacaagactgaatcagcagttcaaggacaaggccaccmtgaccrkrgacacaagcacaagcaccgtgtacatggagctgagcagcctgagaagcgaggacaccgccgtgtactactgcgctagagtgtggagcggcaacgccttcgactactggggccaaggcaccctggtgaccgtgagcagc

SEQ ID NO: 28 - DNA, 339 bp, synthetic construct
  source 1..339: mol_type = other DNA
gacatcgtgatgacacagagccctgacagcctggccgtgagcctgggcgagagagccaccatcaactgcctgagcagccaaagcctgctggctagcggcaatcagagaaactacctgacctggtatcagcagaagcctggacagcctcctaagctgctgatctactgggctagcacaagagacagcggcgtgcctgacagattcascggcagcggcagcggcrysgacttcaccctgaccatcagcagcstgcaagccgaggacgtggccgtgtactactgtcagaacggcttcagcttccctttcaccttcggccaaggcaccaagctggagatcaag

SEQ ID NO: 29 - DNA, 354 bp, synthetic construct
  source 1..354: mol_type = other DNA
caggtccagctgcagcagcctggggctgagctggtgaggcctggaacttcagtgaagctgtcctgcaaggcttctggttattccttcaccaactactgggtgaactgggtgaagcagaggcctggacaaggccttgagtggattggcatgattcatccttccggtagcgaaactagattaaatcagcggttcagggacaagaccacattgactgtagacagatcgtccaatacagcctatatgcaactcaataggccgacatctgaagactctgcggtctattactgtgcaagggtctggtccggtaacgcctttgactactggggccaaggcaccactctcatagtctcctca

SEQ ID NO: 30 - DNA, 339 bp, synthetic construct
  source 1..339: mol_type = other DNA
gacattgtgatgacgcagtctccatcctccctgactgtgacagcaggagagaaggtcactttgaactgcatgtccagtcagagtctgttaaacagtggaaatcaaaaaaactacttgacctggtaccagcagaaaccagggcagcctcctaaattgttgatctattgggcatccactagggattctggggtccctgatcgcttcacaggcggtggatctggagtagatttcactctcaccatctacagggtgcaggctgaagacctggcattttatttctgtcagaatggtttcagttttccattcacgttcggctcggggacaaagttggaaatgaaa

SEQ ID NO: 31 - DNA, 354 bp, synthetic construct
  source 1..354: mol_type = other DNA
caggtccagctgcagcagcctggggctgagctggtgaggcctggaacttcagtgaagctgtcctgcaaggcttctggttattccttcaccaactactggatgaactgggtgaaacagaggcctggacaaggccttgagtggattggcatgattcatccttccggtagcgaaagtaggttaaatcagaagttcaaggacaaggccacattgactgtagacaaatcgtccaatacagcctatatgcaactcaataggccgacatctgcagactctgcggtctattactgtgcaagggtctggtccggtaacgcctttgattactggggccaaggcaccactctcatagtctcctca

SEQ ID NO: 32 - DNA, 339 bp, synthetic construct
  source 1..339: mol_type = other DNA
gacattgtgatgacacagtctccatcctccctgactgtgacagcaggggagaaggtcactttgagctgcatgtccagtcagagtctgttaaacagtggaaatcaaaaaaactacttgacctggtaccagcagaaaccagggcagcctcctaaattgttgatcttttgggcatccactagggattctggggtccctgatcgcttcacaggcggtggatctggaacacttttcactctcaccatctacagggtgcaggctgaagacctggcattttattactgtcagaatggtttcagttttccattcacgttcggctcggggacaaagttggaaatgaga

(19) United States
(12) Patent Application Publication
(10) Pub. No.: US 2025/0136678 A1
(43) Pub. Date: May 1, 2025

(54) ANTIBODIES BINDING CLDN18.2 AND USES THEREOF

(71) Applicant: BIOSION INC., Nanjing (CN)

(72) Inventors: Mingjiu CHEN, Nanjing (CN); Zeyu PENG, Nanjing (CN)

(21) Appl. No.: 18/701,713

(22) PCT Filed: Oct. 19, 2022
(86) PCT No.: PCT/CN2022/126036

§ 371 Date: Apr. 16, 2024

Related U.S. Application Data
(63) Continuation of application No. PCT/CN2021/124767, filed on Oct. 19, 2021.

(30) Foreign Application Priority Data
Oct. 19, 2021 (WO) PCT/CN2021/124767

(51) Int. Cl. C07K 16/28; A61K 47/68

(52) U.S. Cl. CPC C07K 16/28; A61K 47/6817; C07K 2317/14; C07K 2317/24; C07K 2317/565; C07K 2317/732; C07K 2317/77

(57) ABSTRACT

Provided is an isolated monoclonal antibody that specifically binds human CLDN18.2, or the antigen-binding portion thereof. A nucleic acid molecule encoding the antibody or the antigen-binding portion thereof, an expression vector, a host cell and a method for expressing the antibody or the antigen-binding portion thereof are also provided. Further provided are an antibody-drug conjugate and a pharmaceutical composition comprising the antibody or the antigen-binding portion thereof, as well as a treatment method using the anti-CLDN 18.2 antibody or the antigen-binding portion thereof.

### RELATED APPLICATIONS AND INCORPORATION BY REFERENCE

This application claims priority to International Patent Application Serial No. PCT/CN2021/124767 filed Oct. 19, 2021.

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing XML labeled “55532-00041SequenceListingXML” which was created on Oct. 12, 2022 and is 42.4 bytes. The entire content of the sequence listing is incorporated herein by reference in its entirety.

### FIELD OF THE INVENTION

The present disclosure relates generally to an isolated monoclonal antibody, particularly a mouse, chimeric or humanized monoclonal antibody, or an antigen-binding portion thereof, that binds to CLDN18.2, with high affinity and functionality. A nucleic acid molecule encoding the antibody or the antigen-binding portion thereof, an expression vector, a host cell and a method for expressing the antibody or the antigen-binding portion thereof are also provided. The present disclosure further provides a bispecific molecule, an immunoconjugate, a chimeric antigen receptor, and a pharmaceutical composition which may comprise the antibody or the antigen-binding portion thereof, as well as a treatment method using the anti-CLDN18.2 antibody or the antigen-binding portion thereof of the disclosure.

### BACKGROUND OF THE INVENTION

Antibody-based therapy is becoming one of the most promising methods for treating cancer patients, as antibody-based therapeutics may have higher binding specificity and produce lower side effects. However, due to the heterogeneity of malignant tumors, different cancers show different molecular biology characteristics, especially for gastric cancer. The degree of heterogeneity for gastric cancer is relatively high, and the targeted therapeutic drugs developed in the past are basically ineffective or effective for a very small ratio of patients when they are used alone.

Claudins are a family of cell-surface proteins that establish a paracellular barrier and control the flow of molecules between cells, playing critical roles in cell signaling and epithelial cell polarity maintaining (Singh et al., (2010) J Oncol 2010; 541957). Claudin 18 has two splice variants, Claudin18.1/CLDN18.1 and Claudin18.2/CLDN18.2, the latter one is a 27.8 kDa transmembrane protein comprising four membrane spanning domains with two small extracellular loops.

CLDN18.2 has been found to be a promising target for antibody therapy for gastric and esophageal cancers (J Hematol Oncol. 2017(1):105), as there is no detectable expression of CLDN18.2, as measured by e.g., RT-PCR, in normal tissues with exception of stomach. Despite of its unique expression specificity, CLDN18.2 shares an extremely high sequence similarity with CLDN18.1, with a few different amino acid residues at the extracellular domains. Therefore, it is extremely difficult to develop antibodies that target CLDN18.2 only.

Ganymed Pharmaceuticals AG developed a chimeric IgG1 antibody IMAB362 which recognizes the first extracellular domain of CLDN18.2 with high affinity and specificity. However, the chimeric antibody may cause immunogenicity to the patients in clinical use when relatively high doses are required. Therefore, there is a need for additional anti-CLDN18.2 antibodies with lower immunogenicity and higher efficacy.

Citation or identification of any document in this application is not an admission that such document is available as prior art to the present invention.

### SUMMARY OF THE INVENTION

The present disclosure provides an isolated monoclonal antibody, for example, a mouse, chimeric or humanized monoclonal antibody, or an antigen-binding portion thereof, that binds to CLDN18.2 (e.g., the human CLDN18.2) and i) has much higher binding affinity/capability to human CLDN18.2 than prior art anti-CLDN18.2 antibodies such as Zolbetuximab, ii) has no cross-reaction to human CLDN18.1, iii) is internalized into CLDN18.2⁺ cells at higher rates than prior art anti-CLDN18.2 antibodies such as Zolbetuximab, iv) induces higher antibody-dependent cell-mediated cytotoxicity (ADCC) against CLDN18.2⁺ cells than prior art anti-CLDN18.2 antibodies such as Zolbetuximab, and/or v) has in vivo anti-tumor activity.

The antibody or antigen-binding portion of the disclosure can be used for a variety of applications, including treatment of diseases associated with CLDN18.2, such as cancers.

Accordingly, in one aspect, the disclosure pertains to an isolated monoclonal antibody, or an antigen-binding portion thereof, that binds CLDN18.2, having (i) a heavy chain variable region that may comprise a VH CDR1 region, a VH CDR2 region and a VH CDR3 region, wherein the VH CDR1 region, the VH CDR2 region and the VH CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1 (X1=V), 2 (X1=V, X2=T, X3=Q, X4=K) and 3, respectively; (2) SEQ ID NOs: 1 (X1=V), 2 (X1=I, X2=T, X3=R, X4=R) and 3, respectively; or (3) SEQ ID NOs: 1 (X1=M), 2 (X1=1, X2=S, X3=K, X4=K) and 3, respectively; and/or (ii) a light chain variable region that may comprise a VL CDR1 region, a VL CDR2 region and a VL CDR3 region, wherein the VL CDR1 region, the VL CDR2 region and the VL CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 4 (X1=L, X2=N, X3=S, X4=R), 5 and 6, respectively; (2) SEQ ID NOs: 4 (X1=L, X2=A, X3=S, X4=R), 5 and 6, respectively; (3) SEQ ID NOs: 4 (X1=L, X2=N, X3=A, X4=R), 5 and 6, respectively; (4) SEQ ID NOs: 4 (X1=L, X2=N, X3=E, X4=R), 5 and 6, respectively; or (5) SEQ ID NOs: 4 (X1=M, X2=N, X3=S, X4=K), 5 and 6, respectively.

The isolated monoclonal antibody, or the antigen-binding portion thereof, of the present disclosure may comprise a heavy chain variable region having a VH CDR1 region, a VH CDR2 region and a VH CDR3 region, and a light chain variable region having a VL CDR1 region, a VL CDR2 region and a VL CDR3 region, wherein the VH CDR1, VH CDR2, VH CDR3, VL CDR1, VL CDR2 and VL CDR3 may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1 (X1=V), 2 (X1=V, X2=T, X3=Q, X4=K), 3, 4 (X1=L, X2=N, X3=S, X4=R), 5 and 6, respectively; (2) SEQ ID NOs: 1 (X1=V), 2 (X1=V, X2=T, X3=Q, X4=K), 3, 4 (X1=L, X2=A, X3=S, X4=R), 5 and 6, respectively; (3) SEQ ID NOs: 1 (X1=V), 2 (X1=V, X2=T, X3=Q, X4=K), 3, 4 (X1=L, X2=N, X3=A, X4=R), 5 and 6, respectively; (4) SEQ ID NOs: 1 (X1=V), 2 (X1=V, X2=T, X3=Q, X4=K), 3, 4 (X1=L, X2=N, X3=E, X4=R), 5 and 6, respectively; (5) SEQ ID NOs: 1 (X1=V), 2 (X1=I, X2=T, X3=R, X4=R), 3, 4 (X1=M, X2=N, X3=S, X4=K), 5 and 6, respectively; or (6) SEQ ID NOs: 1 (X1=M), 2 (X1=1, X2=S, X3=K, X4=K), 3, 4 (X1=M, X2=N, X3=S, X4=K), 5 and 6, respectively.

The heavy chain variable region may comprise an amino acid sequence having at least 85%, 86%, 87%, 88%, 89%, 90%, 91%, 92%, 93%, 94%, 95%, 96%, 97%, 98%, 99% or 100% identity to 7, 9 (X1=S, X2=I, X3=K, X4=A; X1=S, X2=I, X3=T, X4=V; X1=T, X2=I, X3=T, X4=V; X1=S, X2=M, X3=T, X4=V), 10 (X1=R, X2=A, X3=L, X4=V; X1=K, X2=V, X3=L, X4=V; X1=K, X2=A, X3=M, X4=V; X1=K, X2=A, X3=L, X4=R), 12, or 14. The amino acid sequence of SEQ ID NO: 7 may be encoded by the nucleotide sequences of SEQ ID NOs: 21 and 23, respectively. The amino acid sequences of SEQ ID NOs: 9 (X1=S, X2=I, X3=K, X4=A), 9 (X1=S, X2=I, X3=T, X4=V), 9 (X1=T, X2=I, X3=T, X4=V), 9 (X1=S, X2=M, X3=T, X4=V), 10 (X1=R, X2=A, X3=L, X4=V), 10 (X1=K, X2=V, X3=L, X4=V), 10 (X1=K, X2=A, X3=M, X4=V), 10 (X1=K, X2=A, X3=L, X4=R), 12, or 14 may be encoded by the nucleotide sequences of SEQ ID NOs: 25 (S=G. M=A, R=G, Y=C, S=C), 25 (S=G. M=C, R=A, Y=T, S=G), 25 (S=C, M=C, R=A, Y=T, S=G), 26 (S1=G, R1=A, R2=G, Y=C, S2=C), 26 (S1=C, R1=G, R2=A, Y=C, S2=C), 26 (S1=C, R1=A, R2=G, Y=T, S2=G), 27 (M=A, R1=G, K=T, R2=G), 27 (M=C, R1=A, K=G, R2=A), 29, and 31, respectively.

The light chain variable region may comprise an amino acid sequence having at least 85%, 86%, 87%, 88%, 89%, 90° %, 91%, 92%, 93%, 94%, 95%, 96%, 97%, 98%, 99% or 100% identity to SEQ ID NOs: 8 (X1=N, X2=S; X1=A, X2=S; X1=N, X2=A; X1=N, X2=E), 11 (X1=T, X2=V, X3=V; X1=S, X2=V, X3=V; X1=T, X2=T, X3=V; X1=T, X2=V, X3=L), 13, or 15. The amino acid sequences of SEQ ID NOs: 8 (X1=N, X2=S), 8 (X1=A, X2=S), 8 (X1=N, X2=A), 8 (X1=N, X2=E), 11 (X1=T, X2=V, X3=V), 11 (X1=S, X2=V, X3=V), 11 (X1=T, X2=T, X3=V), 11 (X1=T, X2=V, X3=L), 13, or 15 may be encoded by the nucleotide sequences of SEQ ID NOs: 22, 24 (R1=G, M=C, R2=A, V=G, B=C), 24 (R1=A, M=A, R2=G, V=C, B=T), 24 (R1=A, M=A, R2=G, V=A, B=G), 28 (S1=C, R=G, Y=T, S2=G, S3=G), 28 (S1=G, R=G, Y=T, S2=G, S3=G), 28 (S1=C, R=A, Y=C, S2=C, S3=G), 28 (S1=C, R=G, Y=T, S2=G, S3=C), 30, and 32, respectively.

The isolated monoclonal antibody, or the antigen-binding portion thereof, of the present disclosure may comprise a heavy chain variable region and a light chain variable region, the heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 7 and 8 (X1=N, X2=S), respectively; (2) SEQ ID NOs: 7 and 8 (X1=A, X2=S), respectively; (3) SEQ ID NOs: 7 and 8 (X1=N, X2=A), respectively; (4) SEQ ID NOs: 7 and 8 (X1=N, X2=E), respectively; (5) SEQ ID NOs: 9 (X1=S, X2=I, X3=K, X4=A) and 11 (X1=T, X2=V, X3=V), respectively; (6) SEQ ID NOs: 9 (X1=S, X2=I, X3=T, X4=V) and 11 (X1=T, X2=V, X3=V), respectively; (7) SEQ ID NOs: 9 (X1=T, X2=I, X3=T, X4=V) and 11 (X1=T, X2=V, X3=V), respectively; (8) SEQ ID NOs: 9 (X1=S, X2=M, X3=T, X4=V) and 11 (X1=T, X2=V, X3=V), respectively; (9) SEQ ID NOs: 10 (X1=R, X2=A, X3=L, X4=V) and 11 (X1=T, X2=V, X3=V), respectively; (10) SEQ ID NOs: 10 (X1=K, X2=V, X3=L, X4=V) and 11 (X1=T, X2=V, X3=V), respectively; (11) SEQ ID NOs: 10 (X1=K, X2=A, X3=M, X4=V) and 11 (X1=T, X2=V, X3=V), respectively; (12) SEQ ID NOs: 10 (X1=K, X2=A, X3=L, X4=R) and 11 (X1=T, X2=V, X3=V), respectively; (13) SEQ ID NOs: 9 (X1=S, X2=I, X3=K, X4=A) and 11 (X1=S, X2=V, X3=V), respectively; (14) SEQ ID NOs: 9 (X1=S, X2=I, X3=T, X4=V) and 11 (X1=S, X2=V, X3=V), respectively; (15) SEQ ID NOs: 9 (X1=T, X2=I, X3=T, X4=V) and 11 (X1=S, X2=V, X3=V), respectively; (16) SEQ ID NOs: 9 (X1=S, X2=I, X3=K, X4=A) and 11 (X1=T, X2=T, X3=V), respectively; (17) SEQ ID NOs: 9 (X1=S, X2=I, X3=T, X4=V) and 11 (X1=T, X2=T, X3=V), respectively; (18) SEQ ID NOs: 9 (X1=T, X2=I, X3=T, X4=V) and 11 (X1=T, X2=T, X3=V), respectively; (19) SEQ ID NOs: 9 (X1=S, X2=1, X3=K, X4=A) and 11 (X1=T, X2=V, X3=L), respectively; (20) SEQ ID NOs: 9 (X1=S, X2=1, X3=T, X4=V) and 11 (X1=T, X2=V, X3=L), respectively; (21) SEQ ID NOs: 9 (X1=T, X2=1, X3=T, X4=V) and 11 (X1=T, X2=V, X3=L), respectively; (22) SEQ ID NOs: 12 and 13, respectively; or (23) SEQ ID NOs: 14 and 15, respectively.

The isolated monoclonal antibody, or the antigen-binding portion thereof, of the present disclosure may comprise a heavy chain and a light chain linked by disulfide bonds, the heavy chain may comprise a heavy chain variable region and a heavy chain constant region, the light chain may comprise a light chain variable region and a light chain constant region, wherein the C terminus of the heavy chain variable region is linked to the N terminus of the heavy chain constant region, and the C terminus of the light chain variable region is linked to the N terminus of the light chain constant region, wherein the heavy chain variable region and the light chain variable region may comprise amino acid sequences described above.

The heavy chain constant region may be with enhanced FcR binding affinity, such as human IgG1 constant region having the amino acid sequence set forth in e.g., SEQ ID NO.: 16, or genetically engineered human IgG2 or IgG4 constant region, or a functional fragment thereof. The heavy chain constant region may also be with normal or reduced FcR binding affinity in certain embodiments. The light chain constant region may be human kappa constant region having the amino acid sequences set forth in e.g., SEQ ID NO.: 17.

The antibody of the present disclosure in certain embodiments may comprise or consist of two heavy chains and two light chains, wherein each heavy chain may comprise the heavy chain constant region, heavy chain variable region or CDR sequences mentioned above, and each light chain may comprise the light chain constant region, light chain variable region or CDR sequences mentioned above. The antibody or the antigen-binding portion thereof of the present disclosure in other embodiments may be a single chain variable fragment (scFv) antibody, or antibody fragments, such as Fab or F(ab′)2 fragments.

The disclosure also provides a bispecific molecule that may comprise the antibody, or the antigen-binding portion thereof, of the disclosure, linked to a second functional moiety (e.g., a second antibody) having a different binding specificity than said antibody, or antigen-binding portion thereof. The antibody or the antigen binding portion thereof of the present disclosure can be made into part of a chimeric antigen receptor (CAR). Also provided is an immune cell that may comprise the antigen chimeric receptor, such as a T cell and a NK cell. The antibody or antigen binding portion thereof of the disclosure can also be encoded by or used in conjunction with an oncolytic virus.

The disclosure also provides an immunoconjugate, such as an antibody-drug conjugate, that may comprise an antibody, or antigen-binding portion thereof, of the disclosure, linked to a therapeutic agent, such as a cytotoxin. In certain embodiments, the immunoconjugate comprises an antibody, or antigen-binding portion thereof, of the disclosure conjugated to a toxic recombinant protein. The toxic recombinant protein may be DT3C, having e.g., the amino acid sequence of SEQ ID NO: 20.

The disclosure further provides a nucleic acid molecule encoding the antibody or antigen-binding portion thereof of the disclosure, as well as an expression vector comprising such a nucleic acid molecule and a host cell comprising such an expression vector. A method for preparing the anti-CLDN18.2 antibody or antigen binding portion thereof using the host cell of the disclosure is provided, comprising steps of (i) expressing the antibody or antigen binding portion thereof in the host cell, and (ii) isolating the antibody or antigen binding portion thereof from the host cell or its cell culture.

The disclosure provides a composition comprising the antibody or antigen binding portion thereof, the immunoconjugate, the bispecific molecule, the immune cell, the oncolytic virus, the nucleic acid molecule, the expression vector, or the host cell of the disclosure, and a pharmaceutically acceptable carrier. In certain embodiments, the pharmaceutical composition may further contain a therapeutic agent for treating a specific disease, such as an anti-tumor agent.

In yet another aspect, the disclosure provides a method for treating a disease associated with CLDN18.2, which may comprise administering to a subject a therapeutically effective amount of the composition of the present disclosure.

The disease may be tumor or cancer. The tumor or cancer includes, but not limited to, gastric cancer, esophageal cancer, cancer of the gastroesophageal junction, pancreatic cancer, cancer of the bile duct, lung cancer, ovarian cancer, colon cancer, hepatic cancer, head and neck cancer, or gallbladder cancer. In certain embodiments, the tumor or cancer is gastric cancer. The composition may comprise the antibody, or the antigen-binding portion thereof, with relatively high FcR binding heavy chain constant regions, the bispecific molecule, the immunoconjugate, the immune cell carrying the CAR, the nucleic acid molecule, or the expression vector of the disclosure. In some embodiments, at least one additional anti-cancer antibody can be further administered, such as an anti-VISTA antibody, an anti-PD-L1 antibody, an anti-LAG-3 antibody and/or an anti-CTLA-4 antibody. In yet another embodiment, an antibody, or an antigen-binding portion thereof, of the disclosure is administered with a cytokine (e.g., IL-2 and/or IL-21), or a costimulatory antibody (e.g., an anti-CD137 and/or anti-GITR antibody). The antibody or antigen binding portion of the present disclosure may be, for example, mouse, chimeric or humanized. In certain embodiments, the subject is human.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1C show the binding capability of Zolbetuximab and hIgG to 293T-CLDN18.2 cells (A), the binding capability of ab203563 and Zolbetuximab to BAF3-CLDN18.1 cells (B), and the binding capability of Zolbetuximab and ab203563 to BAF3-CLDN18.2 cells (C) in a cell-based binding FACS assay.

FIGS. 2A-2C show the binding capability of mouse antibodies E1A1F4B5, E1B1B8C7 and E1H4C3E5 to BAF3-CLDN18.2 cells (A), BAF3-CLDN18.1 cells (B) and BAF3 cells (C) in a cell-based binding FACS assay.

FIG. 3 shows the ability of mouse antibodies E1A1F4B5, E1B1B8C7 and E1H4C3E5 to block Zolbetuximab binding to cell surface human CLDN18.2 in a cell-based blocking FACS assay.

FIG. 4 shows the binding capability of chimeric antibodies chE1B1B8C7-V1, chE1B1B8C7-V2 and chE1B1B8C7-V3 to BAF3-CLDN18.2 cells in a cell-based binding FACS assay.

FIGS. 5A-5B show the binding capability of humanized antibodies huE1B1B8C7-V1-huE1B1B8C7-V9 (A) and huE1B1B8C7-V10-huE1B1B8C7-V17 (B) to BAF3-CLDN18.2 cells in a cell-based binding FACS assay.

FIG. 6 shows the internalization-mediated cellular toxicities of antibody (huE1B1B8C7-V12 and huE1B1B8C7-V14)-DT3C conjugates against 293T-CLDN18.2 cells.

FIGS. 7A-7C show the ability of antibodies chE1B1B8C7-V1 and huE1B1B8C7-V12 to induce antibody-dependent cellular cytotoxicity (ADCC) against BAF3-CLDN18.2 cells (A), KATO III cells (B) and 293T-CLDN18.2 cells (C) in vitro.

FIGS. 8A-8C show the cytotoxicity of huE1B1B8C7-V12-toxin conjugates against 293T-CLDN18.2 cells (A), BAF3-CLDN18.2 cells (B) and BAF3-CLDN18.1 cells (C) in vitro.

### DETAILED DESCRIPTION OF THE INVENTION

To ensure that the present disclosure may be more readily understood, certain terms are first defined. Additional definitions are set forth throughout the detailed description.

The term “Claudin18.2” or “CLDN18.2” refers to Claudin-18 splice variant 2 derived from mammals, such as primates (e.g. humans, monkeys) and rodents (e.g. mice). In certain embodiments, CLDN18.2 is human CLDN18.2. Exemplary sequence of human CLDN18.2 includes the one having NCBI Ref Seq No. NP_001002026.1. CLDN18.2 is expressed in a cancer cell. In one embodiment, the CLDN18.2 is expressed on the surface of a cancer cell.

The term “antibody” as used herein refers to an immunoglobulin molecule that recognizes and specifically binds a target, through at least one antigen-binding site wherein the antigen-binding site is usually within the variable region of the immunoglobulin molecule. As used herein, the term encompasses intact polyclonal antibodies, intact monoclonal antibodies, single-chain Fv (scFv) antibodies, heavy chain antibodies (HCAbs), light chain antibodies (LCAbs), multispecific antibodies, bispecific antibodies, monospecific antibodies, monovalent antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding portion” of the intact antibodies. An IgG is a glycoprotein which may comprise two heavy (H) chains and two light (L) chains inter-connected by disulfide bonds. Each heavy chain may be comprised of a heavy chain variable region (abbreviated herein as VH) and a heavy chain constant region. The heavy chain constant region may be comprised of three domains, CH1, CH2 and CH3. Each light chain may be comprised of a light chain variable region (abbreviated herein as VL) and a light chain constant region. The light chain constant region may be comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment which may comprise two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR); and (viii) a nanobody, a heavy chain variable region containing a single variable domain and two constant domains. Furthermore, although the two domains of the Fv fragment. VL and VH, are coded by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a CLDN18.2 protein is substantially free of antibodies that specifically bind antigens other than CLDN18.2 proteins). An isolated antibody that specifically binds a human CLDN18.2 protein may, however, have cross-reactivity to other antigens, such as CLDN18.2 proteins from other species. Moreover, an isolated antibody can be substantially free of other cellular material and/or chemicals.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human CLDN18.2” is intended to refer to an antibody that binds to human CLDN18.2 protein (and possibly a CLDN18.2 protein from one or more non-human species) but does not substantially bind to non-CLDN18.2 proteins. Preferably, the antibody binds to human CLDN18.2 protein with “high affinity”, namely with a K_{D} of 5.0×10⁻⁸ M or less, more preferably 1.0×10⁻⁸ M or less, and more preferably 5.0×10⁻⁹ M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” for an IgG antibody refers to an antibody having a K_{D} of 1.0×10⁻⁷ M or less, more preferably 1.0×10⁻⁸ M or less, even more preferably 1.0×10⁻⁹ M or less, and even more preferably 1.0×10⁻¹⁰ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody or the antigen binding portion of the present disclosure sufficient to prevent or ameliorate the symptoms associated with a disease or condition and/or lessen the severity of the disease or condition. A therapeutically effective amount is understood to be in context to the condition being treated, where the actual effective amount is readily discerned by those of skill in the art.

The term “antibody-dependent cell-mediated cytotoxicity” or “ADCC” is a mechanism of cell-mediated immune defense whereby an effector cell of the immune system actively lyses a target cell, whose membrane-surface antigens have been bound by the anti-CLDN18.2 antibodies.

Various aspects of the disclosure are described below in further detail.

The antibody or antigen binding portion thereof of the disclosure may be mouse, chimeric or humanized. The antibody or antigen binding portion thereof of the disclosure is structurally and chemically characterized below. The amino acid sequence ID numbers of the heavy/light chain variable regions and CDRs of the antibodies or antigen binding portions thereof of the disclosure are summarized in Table 1 below, some antibodies sharing the same VH or VL. The antibody of the disclosure may comprise human IgG1 heavy chain constant region and/or human kappa light chain constant region.

The heavy chain variable region CDRs and the light chain variable region CDRs in Table 1 have been defined by the Kabat numbering system. However, as is well known in the art, CDR regions can also be determined by other systems such as Chothia, and IMGT, AbM, or Contact numbering system/method, based on heavy chain/light chain variable region sequences.

Accordingly, in one embodiment, an antibody of the disclosure, or an antigen binding portion thereof, may comprise:
(a) a heavy chain variable region which may comprise an amino acid sequence listed above in Table 1; and
(b) a light chain variable region which may comprise an amino acid sequence listed above in Table 1, or the V_{L} of another Anti-CLDN18.2 antibody, wherein the antibody specifically binds human CLDN18.2.

In another embodiment, an antibody of the disclosure, or an antigen binding portion thereof, may comprise:
(a) the CDR1, CDR2, and CDR3 regions of the heavy chain variable region listed above in Table 1; and
(b) the CDR1, CDR2, and CDR3 regions of the light chain variable region listed above in Table 1 or the CDRs of another anti-CLDN18.2 antibody, wherein the antibody specifically binds human CLDN18.2.

Accordingly, in one embodiment, the antibody may comprise a heavy chain variable region which may comprise CDR1, CDR2, and CDR3 sequences and/or a light chain variable region which may comprise CDR1, CDR2, and CDR3 sequences, wherein:
(a) the heavy chain variable region CDR1 sequence may comprise a sequence listed in Table 1 above, and/or conservative modifications thereof; and/or
(b) the heavy chain variable region CDR2 sequence may comprise a sequence listed in Table 1 above, and/or conservative modifications thereof; and/or
(c) the heavy chain variable region CDR3 sequence may comprise a sequence listed in Table 1 above, and conservative modifications thereof; and/or
(d) the light chain variable region CDR1, and/or CDR2, and/or CDR3 sequences may comprise the sequence(s) listed in Table 1 above; and/or conservative modifications thereof; and
(e) the antibody specifically binds human CLDN18.2.
   In various embodiments, the antibody can be, for example, a mouse, human, humanized or chimeric antibody.

TABLE 1
Amino acid sequence ID numbers of heavy/light chain variable regions and CDRs

| | VH | | | | VL | | | |
|---|---|---|---|---|---|---|---|---|
| Antibody ID | CDR1 | CDR2 | CDR3 | VH | CDR1 | CDR2 | CDR3 | VL |
| mouse | 1, X1 = V | 2, X1 = V, X2 = T, | 3 | 7 | 4, X1 = L, X2 = N, | 5 | 6 | 8, X1 = N, X2 = S |
| E1B1B8C7 | | X3 = Q, X4 = K | | | X3 = S, X4 = R | | | |
| chE1B1B8C7-V1 | 1, X1 = V | 2, X1 = V, X2 = T, | 3 | 7 | 4, X1 = L, X2 = A, | 5 | 6 | 8, X1 = A, X2 = S |
| | | X3 = Q, X4 = K | | | X3 = S, X4 = R | | | |
| chE1B1B8C7-V2 | 1, X1 = V | 2, X1 = V, X2 = T, | 3 | 7 | 4, X1 = L, X2 = N, | 5 | 6 | 8, X1 = N, X2 = A |
| | | X3 = Q, X4 = K | | | X3 = A, X4 = R | | | |
| chE1B1B8C7-V3 | 1, X1 = V | 2, X1 = V, X2 = T, | 3 | 7 | 4, X1 = L, X2 = N, | 5 | 6 | 8, X1 = N, X2 = E |
| | | X3 = Q, X4 = K | | | X3 = E, X4 = R | | | |
| huE1B1B8C7-V1 | 1, X1 = V | 2, X1 = V, X2 = T, | 3 | 9, X1 = S, X2 = I, | 4, X1 = L, X2 = A, | 5 | 6 | 11, X1 = T, X2 = V, |
| | | X3 = Q, X4 = K | | X3 = K, X4 = A | X3 = S, X4 = R | | | X3 = V |
| huE1B1B8C7-V2 | 1, X1 = V | 2, X1 = V, X2 = T, | 3 | 9, X1 = S, X2 = I, | 4, X1 = L, X2 = A, | 5 | 6 | 11, X1 = T, X2 = V, |
| | | X3 = Q, X4 = K | | X3 = T, X4 = V | X3 = S, X4 = R | | | X3 = V |
| huE1B1B8C7-V3 | 1, X1 = V | 2, X1 = V, X2 = T, | 3 | 9, X1 = T, X2 = I, | 4, X1 = L, X2 = A, | 5 | 6 | 11, X1 = T, X2 = V, |
| | | X3 = Q, X4 = K | | X3 = T, X4 = V | X3 = S, X4 = R | | | X3 = V |
| huE1B1B8C7-V4 | 1, X1 = V | 2, X1 = V, X2 = T, | 3 | 9, X1 = S, X2 = M, | 4, X1 = L, X2 = A, | 5 | 6 | 11, X1 = T, X2 = V, |
| | | X3 = Q, X4 = K | | X3 = T, X4 = V | X3 = S, X4 = R | | | X3 = V |
| huE1B1B8C7-V5 | 1, X1 = V | 2, X1 = V, X2 = T, | 3 | 10, X1 = R, X2 = A, | 4, X1 = L, X2-A, | 5 | 6 | 11, X1 = T, X2 = V, |
| | | X3 = Q, X4 = K | | X3 = L, X4 = V | X3 = S, X4 = R | | | X3 = V |
| huE1B1B8C7-V6 | 1, X1 = V | 2, X1 = V, X2 = T, | 3 | 10, X1 = K, X2 = V, | 4, X1 = L, X2 = A, | 5 | 6 | 11, X1 = T, X2 = V, |
| | | X3 = Q, X4 = K | | X3 = L, X4 = V | X3 = S, X4 = R | | | X3 = V |
| huE1B1B8C7-V7 | 1, X1 = V | 2, X1 = V, X2 = T, | 3 | 10, X1 = K, X2 = A, | 4, X1 = L, X2 = A, | 5 | 6 | 11, X1 = T, X2 = V, |
| | | X3 = Q, X4 = K | | X3 = M, X4 = V | X3 = S, X4 = R | | | X3 = V |
| huE1B1B8C7-V8 | 1, X1 = V | 2, X1 = V, X2 = T, | 3 | 10, X1 = K, X2 = A, | 4, X1 = L, X2 = A, | 5 | 6 | 11, X1 = T, X2 = V, |
| | | X3 = Q, X4 = K | | X3 = L, X4 = R | X3 = S, X4 = R | | | X3 = V |
| huE1B1B8C7-V9 | 1, X1 = V | 2, X1 = V, X2 = T, | 3 | 9, X1 = S, X2 = I, | 4, X1 = L, X2 = A, | 5 | 6 | 11, X1 = S, X2 = V, |
| | | X3 = Q, X4 = K | | X3 = K, X4 = A | X3 = S, X4 = R | | | X3 = V |
| huE1B1B8C7- | 1, X1 = V | 2, X1 = V, X2 = T, | 3 | 9, X1 = S, X2 = I, | 4, X1-L, X2 = A, | 5 | 6 | 11, X1 = S, X2 = V, |
| V10 | | X3 = Q, X4 = K | | X3 = T, X4 = V | X3 = S, X4 = R | | | X3 = V |
| huE1B1B8C7- | 1, X1 = V | 2, X1 = V, X2 = T, | 3 | 9, X1 = T, X2 = I, | 4, X1 = L, X2 = A, | 5 | 6 | 11, X1 = S, X2 = V, |
| V11 | | X3 = Q, X4 = K | | X3 = T, X4 = V | X3 = S, X4 = R | | | X3 = V |
| huE1B1B8C7- | 1, X1 = V | 2, X1 = V, X2 = T, | 3 | 9, X1 = S, X2 = I, | 4, X1 = L, X2 = A, | 5 | 6 | 11, X1 = T, X2 = T, |
| V12 | | X3 = Q, X4 = K | | X3 = K, X4 = A | X3 = S, X4 = R | | | X3 = V |
| huE1B1B8C7- | 1, X1 = V | 2, X1 = V, X2 = T, | 3 | 9, X1 = S, X2 = I, | 4, X1 = L, X2 = A, | 5 | 6 | 11, X1 = T, X2 = T, |
| V13 | | X3 = Q, X4 = K | | X3 = T, X4 = V | X3 = S, X4 = R | | | X3 = V |
| huE1B1B8C7- | 1, X1 = V | 2, X1 = V, X2 = T, | 3 | 9, X1 = T, X2 = I, | 4, X1 = L, X2 = A, | 5 | 6 | 11, X1 = T, X2 = T, |
| V14 | | X3 = Q, X4 = K | | X3 = T, X4 = V | X3 = S, X4 = R | | | X3 = V |
| huE1B1B8C7- | 1, X1 = V | 2, X1 = V, X2 = T, | 3 | 9, X1 = S, X2 = I, | 4, X1 = L, X2 = A, | 5 | 6 | 11, X1 = T, X2 = V, |
| V15 | | X3 = Q, X4 = K | | X3 = K, X4 = A | X3 = S, X4 = R | | | X3 = L |
| huE1B1B8C7- | 1, X1 = V | 2, X1 = V, X2 = T, | 3 | 9, X1 = S, X2 = I, | 4, X1 = L, X2 = A, | 5 | 6 | 11, X1 = T, X2 = V, |
| V16 | | X3 = Q, X4 = K | | X3 = T, X4 = V | X3 = S, X4 = R | | | X3 = L |
| huE1B1B8C7- | 1, X1 = V | 2, X1 = V, X2 = T, | 3 | 9, X1 = T, X2 = I, | 4, X1 = L, X2 = A, | 5 | 6 | 11, X1 = T, X2 = V, |
| V17 | | X3 = Q, X4 = K | | X3 = T, X4 = V | X3 = S, X4 = R | | | X3 = L |
| E1A1F4B5 | 1, X1 = V | 2, X1 = I, X2 = T, | 3 | 12 | 4, X1 = M, X2 = N, | 5 | 6 | 13 |
| | | X3 = R, X4 = R | | | X3 = S, X4 = K | | | |
| E1H4C3E5 | 1, X1 = M | 2, X1 = I, X2 = S, | 3 | 14 | 4, X1 = M, X2 = N, | 5 | 6 | 15 |
| | | X3 = K, X4 = K | | | X3 = S, X4 = K | | | |

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

Antibodies of the disclosure can be prepared using an antibody having one or more of the VH/VL sequences of the anti-CLDN18.2 antibody of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the disclosure pertains to an isolated monoclonal antibody, or antigen binding portion thereof, which may comprise a heavy chain variable region that may comprise CDR1, CDR2, and CDR3 sequences which may comprise the sequences of the present disclosure, as described above, and/or a light chain variable region which may comprise CDR1, CDR2, and CDR3 sequences which may comprise the sequences of the present disclosure, as described above. While these antibodies contain the V_{H} and V_{L} CDR sequences of the monoclonal antibody of the present disclosure, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG-0010109, NT-024637 & BC070333), 3-33 (NG-0010109 & NT-024637) and 3-7 (NG-0010109 & NT-024637). As another example, the following heavy chain germline sequences found in the HCo12 HuMAb mouse are available in the accompanying Genbank Accession Nos.: 1-69 (NG-0010109, NT-024637 & BC070333), 5-51 (NG-0010109 & NT-024637), 4-34 (NG-0010109 & NT-024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the disclosure are those that are structurally similar to the framework sequences used by antibodies of the disclosure. The V_{H} CDR1. CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Accordingly, in another embodiment, the disclosure provides isolated anti-CLDN18.2 monoclonal antibodies, or antigen binding portions thereof, which may comprise a heavy chain variable region that may comprise: (a) a V_{H} CDR1 region which may comprise the sequence of the present disclosure, or an amino acid sequence having one, two, three, four or five amino acid substitutions, deletions or additions; (b) a V_{H} CDR2 region which may comprise the sequence of the present disclosure, or an amino acid sequence having one, two, three, four or five amino acid substitutions, deletions or additions; (c) a V_{H} CDR3 region which may comprise the sequence of the present disclosure, or an amino acid sequence having one, two, three, four or five amino acid substitutions, deletions or additions; (d) a V_{L} CDR1 region which may comprise the sequence of the present disclosure, or an amino acid sequence having one, two, three, four or five amino acid substitutions, deletions or additions; (e) a V₁. CDR2 region which may comprise the sequence of the present disclosure, or an amino acid sequence having one, two, three, four or five amino acid substitutions, deletions or additions; and (f) a V_{L} CDR3 region which may comprise the sequence of the present disclosure, or an amino acid sequence having one, two, three, four or five amino acid substitutions, deletions or additions.

Engineered antibodies of the disclosure include those in which modifications have been made to framework residues within V_{H} and/or V_{L}, e.g. to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure can be chemically modified (e.g., one or more chemical moieties can be attached to the antibody) or be modified to alter its glycosylation, again to alter one or more functional properties of the antibody.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase or reduce the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (α (1, 6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1, 6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. Methods for production of antibodies in a plant system are disclosed in the U.S. patent application corresponding to Alston & Bird LLP attorney docket No. 040989/314911, filed on Aug. 11, 2006. The fucose residues of the antibody can be cleaved off using a fucosidase enzyme; e.g., the fucosidase α-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EP 0 154 316 and EP 0 401 384.

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence.

In a preferred embodiment, the antibodies do not contain asparagine isomerism sites. The deamidation of asparagine may occur on N-G or D-G sequences and result in the creation of an isoaspartic acid residue that introduces a link into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG1 antibody typically falls within the pH range of 6-8. There is speculation that antibodies with a pI outside the normal range may have some unfolding and instability under in vivo conditions. Thus, it is preferred to have an anti-CLDN18.2 antibody that contains a pI value that falls in the normal range. This can be achieved either by selecting antibodies with a pI in the normal range or by mutating charged surface residues.

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and V_{L} sequences of the CLDN18.2 monoclonal antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al. (1988) Proc. Natl. Acad. Sci. USA 85:5879-5883; McCafferty et al., (1990) Nature 348:552-554).

Monoclonal antibodies (mAbs) of the present disclosure can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

Antibodies of the disclosure also can be produced in a host cell transfectoma using, for example, a combination of recombinant DNA techniques and gene transfection methods as is well known in the art (e.g., Morrison. S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338.841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In another aspect, the present disclosure features bispecific molecules which may comprise one or more antibodies of the disclosure linked to at least one other functional molecule, e.g., another peptide or protein (e.g., another antibody or ligand for a receptor) to generate a bispecific molecule that binds to at least two different binding sites or target molecules. Thus, as used herein, “bispecific molecule” includes molecules that have three or more specificities.

Bispecific molecules may be in many different formats and sizes. At one end of the size spectrum, a bispecific molecule retains the traditional antibody format, except that, instead of having two binding arms of identical specificity, it has two binding anns each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

Antibodies of the disclosure can be conjugated to a therapeutic agent to form an immunoconjugate such as an antibody-drug conjugate (ADC). Suitable therapeutic agents include an anti-inflammatory agent and an anti-cancer agent. In the ADC, the antibody and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Patent Publications 20060024317; 20060004081; and 20060247295; the disclosures of which are incorporated herein by reference. In certain embodiments, the anti-CLDN18.2 antibody or antigen binding portion thereof may be conjugated to a toxic recombinant protein. The toxic recombinant protein may be DT3C having e.g., the amino acid sequence of SEQ ID NO: 20.

In another aspect, the present disclosure provides a pharmaceutical composition comprising the antibody or antigen binding portion thereof, the immunoconjugate, the bispecific molecule, the immune cell carrying the chimeric antigen receptor, the oncolytic virus, the nucleic acid molecule, the expression vector, and/or the host cell of the present disclosure formulated together with a pharmaceutically acceptable carrier. The composition may optionally contain one or more additional pharmaceutically active ingredients, such as an anti-tumor agent, an anti-infective agent, or an agent for immunity enhancement. The pharmaceutical composition of the disclosure may be administered in a combination therapy with, for example, an anti-tumor agent, an anti-infective agent, or an agent for immunity enhancement.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from about 0.0001 to 100 mg/kg, and more usually 0.01 to 5 mg/kg, of the host body weight.

A “therapeutically effective dosage” of an anti-CLDN18.2 antibody of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic antibody can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems. J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies or antigen binding portions thereof of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995) Am. J. Physiol. 1233:134; Schreier et al., (1994) J. Biol. Chem. 269:9090; Keinanen and Laukkanen (1994) FEBS Lett. 346:123; and Killion and Fidler (1994) Immunomethods 4:273.

The disclosure provides a method for treating tumor or cancer, which may comprise administering to a subject a therapeutically effective amount of the composition of the present disclosure.

The tumor or cancer includes, but not limited to, gastric cancer, esophageal cancer, cancer of the gastroesophageal junction, pancreatic cancer, cancer of the bile duct, lung cancer, ovarian cancer, colon cancer, hepatic cancer, head and neck cancer, or gallbladder cancer. The composition comprises the antibody, or the antigen-binding portion thereof, with FcR binding heavy chain constant regions, the bispecific molecule, the immunoconjugate, the immune cell carrying the CAR, the nucleic acid molecule, the expression vector or the host cell of the disclosure. In certain embodiments, the subject is human.

In yet another aspect, the disclosure provides methods of combination therapy in which the pharmaceutical composition of the present disclosure is co-administered with one or more additional antibodies that are effective in inhibiting tumor growth in a subject.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Generation of CLDN18.1- and CLDN18.2-Expressing Cell Lines

BAF3-CLDN18.1 and BAF3-CLDN18.2 cells respectively over-expressing human CLDN18.1 (Uniprot No:P56856) and human CLDN18.2 (NP_001002026.1) were prepared, following the manual of lipofectamine 3000 transfection reagent (Thermo Fisher), by transfecting BAF3 cells (Cat #iCcll-m007, iCell Bioscience Inc.) with PCMV-T-P plasmids inserted with CLDN18.1 and CLDN18.2 coding sequences respectively, to obtain stable cell lines, wherein the PCMV-T-P plasmids were constructed by inserting the primycin resistance gene into the vector pCMV-C-His. The expression of human CLDN18.2 was detected by Zolbetuximab (in house made with heavy chain and light chain amino acid sequences of SEQ ID NOs: 18 and 19) which can bind to human CLDN18.2 specifically. The expression of human CLDN18.1 was detected by anti-CLDN18 antibody (Cat #ab203563, Abeam).

Another cell line, 293T-CLDN18.2 over-expressing human CLDN18.2, was purchased from KYinno, Cat #KC-0986.

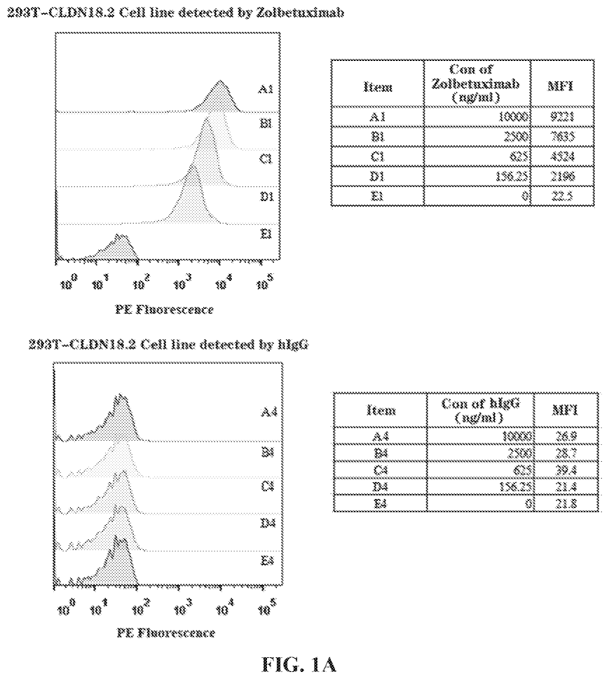

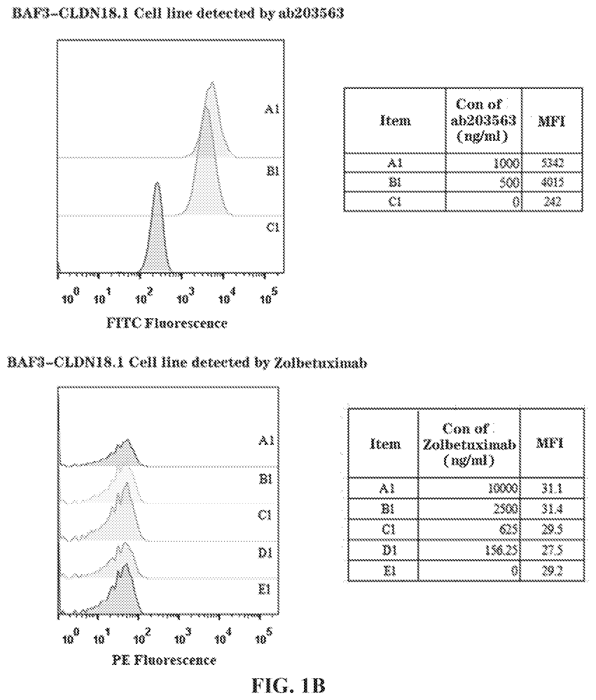

The above three stable cell lines were tested for CLDN18.1 or CLDN18.2 expression using Zolbetuximab, ab203563 and hIgG in flow cytometry (FACS), the results were shown in FIGS. 1A-1C.

The results indicated that all the three cell lines expressed target proteins at high levels.

### Example 2. Generation of Mouse Anti-CLDN18.2 Monoclonal Antibodies Using Hybridoma Technology

### Immunization

Mice aged 6-8 weeks were selected and fed for one week, and then female Balb/C mice and female A/J mice (Shanghai Sippe-Bk Lab Animal) were selected for immunization. 293T-CLDN18.2 cells were collected, re-suspended in PBS with a cell density at 4×10⁸ cells/ml, and injected subcutaneously to the mice using the multiple point injection method. Each mouse was injected with 200 μL cell suspension, 50 μl/point, 8×10⁷ cells in total. The mice were boosted for 3 to 4 times depending on the anti-sera titers. Mice with good titers were given a final boost before hybridoma fusion.

### Hybridoma Fusion and Screening

Cells of murine myeloma cell line (SP2/0-Ag14, ATCC #CRL-1581) were cultured to reach the log phase stage right before hybridoma fusion. Spleen cells from immunized mice were prepared sterilely and fused with murine myeloma cells according to the method as described in Kohler G, and Milstein C, “Continuous cultures of fused cells secreting antibody of predefined specificity,” Nature, 256: 495497(1975). Fused “hybrid cells” were subsequently dispensed into 96-well plates in DMEM/20% FCS/HAT media. Surviving hybridoma colonies were observed under the microscope seven to ten days post fusion. After two weeks, the supernatant from each well was screened in the BAF3-CLDN18.2 cell binding test. Selected hybridoma clones were subcloned by limited dilution to ensure the clonality of the cell line, and then monoclonal antibodies were purified. Briefly, Protein A sepharose column (Cat #AA0273, bestchrom (Shanghai) Biosciences) was washed using PBS buffer in 5 to 10 column volumes. Cell supernatants containing the hybridomas were passed through the columns, and then the columns were washed using PBS buffer until the absorbance for protein reached the baseline. The columns were eluted with elution buffer (0.1 M Glycine-HCl, pH 2.7), and immediately collected into tubes with neutralizing buffer (1 M Tris-HCl, pH 9.0). Fractions containing immunoglobulins were pooled and dialyzed in PBS overnight at 4° C.

Subsequently, in vitro and in vivo functional activity of purified mouse monoclonal antibodies were characterized as follows. Zolbetuximab (IMAB362, Ganymed) was used as Benchmark, in house made with heavy chain and light chain amino acid sequences of SEQ ID NOs: 18 and 19, and in house made anti-CD22 antibody was used as a negative control.

### Example 3. Binding Activity of Mouse Anti-CLDN18.2 Antibodies

The binding activity of mouse anti-CLDN18.2 antibodies of the disclosure to CLDN18.2 or CLDN18.1 was further determined by cell-based binding FACS.

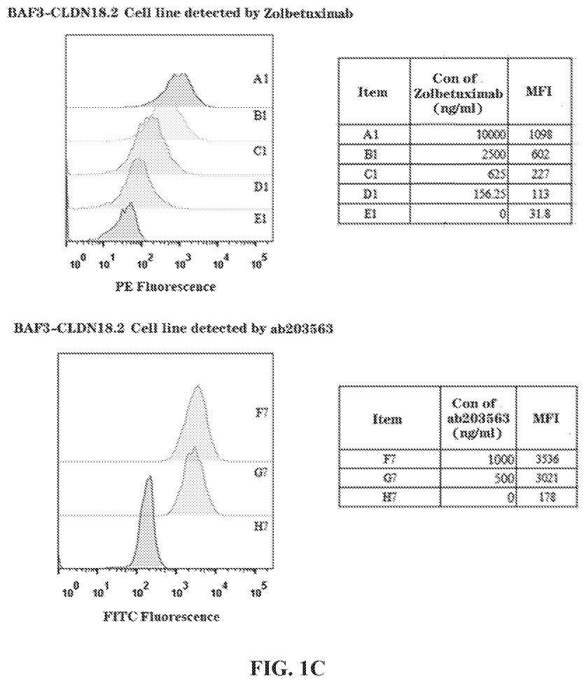

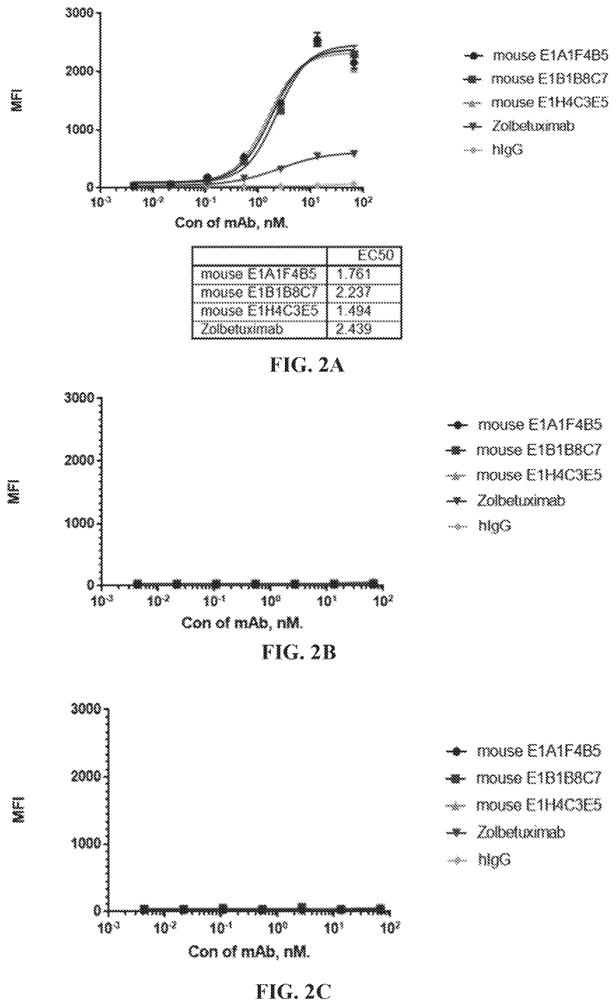

The binding activity of the mouse anti-CLDN18.2 antibodies to human CLDN18.2 or CLDN18.1 expressed on cell surface was tested using BAF3-CLDN18.2 and BAF3-CLDN18.1 cells prepared in Example 1. The BAF3-CLDN18.2, BAF3-CLDN18.1 and BAF3 cells were harvested, washed twice and re-suspended in phosphate buffered saline (PBS) containing 2% v/v Fetal Bovine Serum (FACS buffer), BAF3 cells were used here as blank control. The cells, 1×10⁵ per well, were incubated in 96 well-plates with 100 μl serially diluted antibodies or controls (starting from 10 μg/mL, 5-fold serial dilution) in FACS buffer for 50 minutes on ice. Cells were washed twice with FACS buffer, and 100 μl GAM-PE or GAH-PE (1:1000 dilution in FACS buffer, Cat #115-116-146, Cat #109-115-098, Jackson ImmunoResearch) was added. Following an incubation of 50 minutes at 4° C. in dark, cells were washed three times and re-suspended in FACS buffer. Fluorescence was measured using a Becton Dickinson FACS Canto II-HTS equipment, and the MFI (mean fluorescence intensity) was plotted against antibody concentration. Data was analyzed using Graphpad Prism and EC₅₀ values were reported. The results were shown in FIGS. 2A-2C.

It can be seen from FIG. 2A that the mouse anti-CLDN18.2 antibodies of the disclosure specifically bound to human CLDN18.2 with higher Bmax (maximal binding) and lower EC₅₀ than Zolbetuximab.

It can be seen from FIG. 2B and FIG. 2C that the mouse anti-CLDN18.2 antibodies of the disclosure did not bind to BAF3-CLDN18.1 or BAF3 cells, suggesting that they may not bind proteins other than human CLDN18.2.

### Example 4. Blocking Activity of Mouse Anti-CLDN18.2 Antibodies on CLDN18.2-Benchmark Binding

The activity of the anti-CLDN18.2 antibodies of the disclosure to block benchmark binding to cell surface CLDN18.2 was evaluated by cell-based FACS, using the BAF3-CLDN18.2 cell line prepared in Example 1.

The anti-CLDN18.2 antibodies of the disclosure, and the controls were diluted with FACS buffer starting from 10 μg/mL with 5-fold serial dilution. BAF3-CLDN18.2 cells were harvested from cell culture flasks at the log phase, washed twice and re-suspended in PBS containing 2% v/v Fetal Bovine Serum (FACS buffer). BAF3-CLDN18.2 cells, 1×10⁵ cells per well, were incubated in 96 well-plates with 100 μl/well of diluted anti-CLDN18.2 antibodies or Zolbetuximab for 40 minutes at 4° C., and then added and incubated with biotin labeled Zolbetuximab for 40 minutes at 4° C. Then the cells were washed twice with FACS buffer, added with 100 μl/well SA-PE (1:200 dilution in FACS buffer, Cat #:016-110-084, Jackson Immunoresearch), and incubated for 40 minutes at 4° C. in dark. Cells were washed twice and re-suspended in FACS buffer. Fluorescence was measured using a Becton Dickinson FACS Canto II-HTS equipment. Data was analyzed using Graphpad Prism and IC₅₀ values were reported. The result was shown in FIG. 3.

FIG. 3 showed that the anti-CLDN18.2 antibodies of the disclosure were able to block Zolbetuximab binding to cell surface CLDN18.2, suggesting that these antibodies might bind to the same or similar epitopes as Zolbetuximab did.

### Example 5. Generation and Characterization of Chimeric Antibodies

The heavy/light chain variable regions of the anti-CLDN18.2 mAbs of the disclosure were sequenced, and the sequence ID numbers were summarized in Table 1.

The heavy and light chain variable regions of the anti-CLDN18.2 mouse mAb E1B1B8C7 were cloned in frame to human IgG1 heavy-chain constant region (SEQ ID NO: 16) and human kappa light-chain constant region (SEQ ID NO: 17), respectively, wherein the C terminus of the variable region was linked to the N terminus of the respective constant region. To avoid potential deamidation, the CDR1 sequence of the light chain was further optimized, and the differences between mouse E1B1B8C7 and chE1B1B8C7-V1-chE1B1B8C7-V3 were summarized in Table 1.

The vectors each containing a nucleotide encoding a heavy chain variable region linked to human IgG1 heavy-chain constant region, and the vectors each containing a nucleotide encoding a light chain variable region linked to human kappa light-chain constant region were transiently transfected into 50 ml of 293F suspension cell cultures at a ratio of 1.1:1 light to heavy chain construct, with 1 mg/mL PEI.

Cell supernatants containing the chimeric antibodies were harvested after six days in shaking flasks, and then chimeric antibodies were purified from the cell supernatants. The purified chimeric antibodies were tested in cell-based binding FACS assay following the protocol in Example 3.

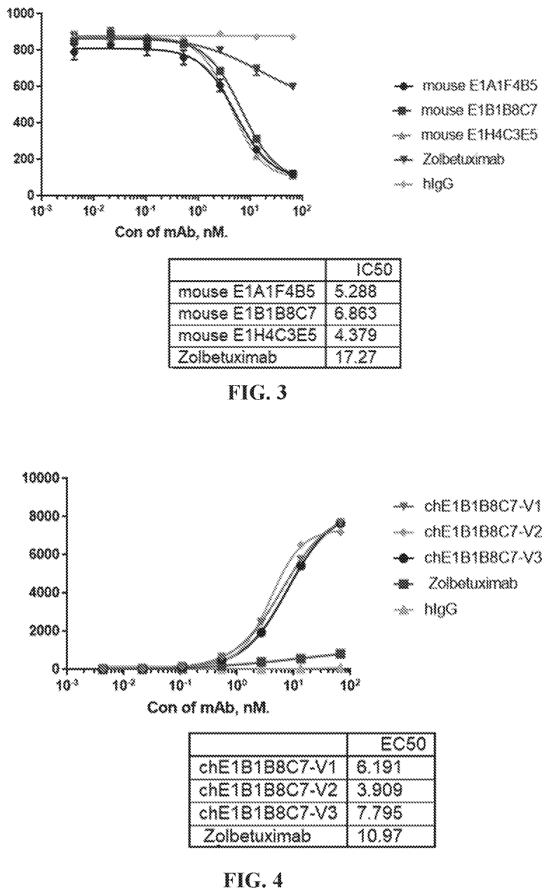

The result was shown in FIG. 4.

According to FIG. 4, the chimeric antibodies chE1B1B8C7-V1, chE1B1B8C7-V2 and chE1B1B8C7-V3 were able to bind to human CLDN18.2 with higher Bmax (maximal binding) and lower EC₅₀ than Zolbetuximab.

### Example 6. Humanization of Anti-CLDN18.2 Monoclonal Antibody E1B1B8C7

The mouse anti-CLDN18.2 antibody E1B1B8C7 was humanized and further characterized. Humanization was conducted using the well-established CDR-grafting method as described in detail below.

To select acceptor frameworks for humanization of the mouse antibody E1B1B8C7, the light and heavy chain variable region sequences of mouse E1B1B8C7 antibody were blasted against the human immunoglobulin gene database. The human germlines with the highest homology were selected as the acceptor frameworks for humanization. The mouse antibody heavy/light chain variable region CDRs were inserted into the selected frameworks, and the residue(s) in the frameworks was/were further back-mutated to obtain more candidate heavy chain/light chain variable regions. A total of 17 exemplary humanized E1B1B8C7 antibodies, namely huE1B1B8C7-V1 to huE1B1B8C7-V17 were obtained, with the light chain variable region CDR1 of SEQ ID NO: 4 (X1=L, X2=A, X3=S, X4=R), whose heavy/light chain variable region sequence ID numbers were set forth in Table 1.

The vectors each containing a nucleotide encoding a humanized heavy chain variable region linked to human IgG1 heavy-chain constant region (SEQ ID NO: 16), and the vectors each containing a nucleotide encoding a humanized light chain variable region linked to human kappa light-chain constant region (SEQ ID NO: 17) were transiently transfected into 50 ml of 293F suspension cell cultures in a ratio of 1.1:1 light to heavy chain construct, with 1 mg/mL PEI.

### Example 7. Characterization of Humanized E1B1B8C7 Antibodies

Cell supernatants containing humanized E1B1B8C7 antibodies were harvested in shaking flasks after six days, and then purified humanized antibodies were obtained by protein A affinity purification and tested in cell-based binding FACS following the protocol in Example 3.

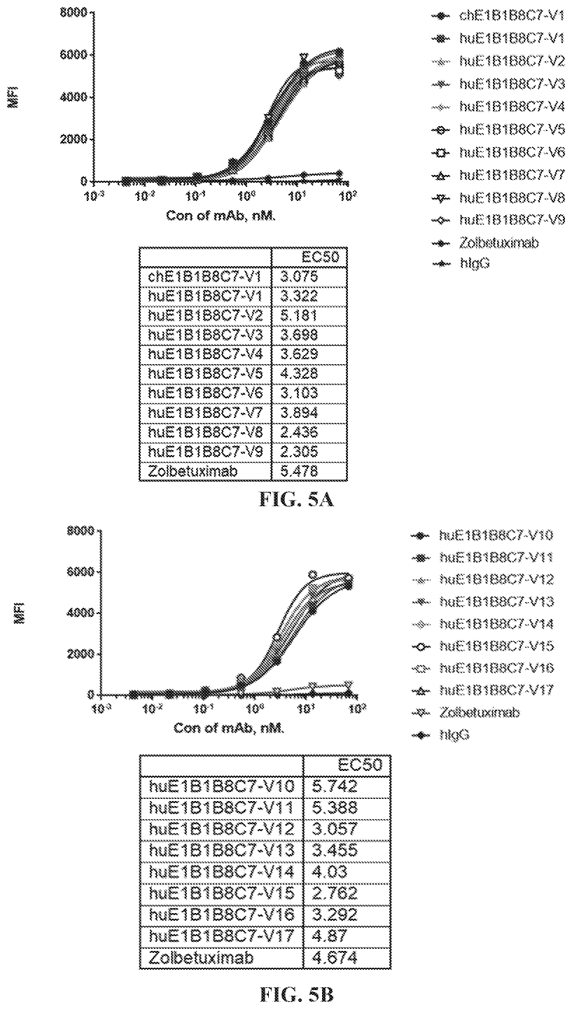

The results were shown in FIGS. 5A-5B.

The data indicated that all the humanized CLDN18.2 antibodies had similar human CLDN18.2 binding activity to the chimeric antibody chE1B1B8C7-V1, which were much higher than that of Zolbetuximab.

The humanized antibodies huE1B1B8C7-V12 and huE1B1B8C7-V14 were further tested for their thermal stabilities. Briefly, a protein thermal shift assay was used to determine Tm (melting temperature) using a GloMelt™ Thermal Shift Protein Stability Kit (Cat #:33022-T, Biotium). Briefly, the GloMelt™ dye was allowed to thaw and reach room temperature. The vial containing the dye was vortexed and centrifuged. Then, 10× dye was prepared by adding 5 μL 200× dye to 95 μL PBS. 2 μL 10× dye and 10 μg humanized antibodies were added, and PBS was added to a total reaction volume of 20 μL. The tubes containing the dye and antibodies were briefly spun and placed in real-time PCR thermocycler (Roche, LightCycler 480 II) set up with a melt curve program having the parameters in Table 2.

TABLE 2
Parameters for Melt Curve Program

| Profile step | Temperature | | Ramp rate | Holding Time |
|---|---|---|---|---|
| Initial hold | 25° | C. | NA | 30 s |
| Melt curve | 25-99° | C. | 0.1° C./s | NA |

The results were shown in Table 3, suggesting that the two antibodies were probably stab e in human body.

TABLE 3
Melting temperatures of anti-CLDN18.2 antibodies

Tm (melting temperature) ° C.

| mAb ID# | Tm1 | Tm2 |
|---|---|---|
| huE1B1B8C7-V12 | 68.5 | 83.0 |
| huE1B1B8C7-V14 | 68.5 | 83.5 |

### Example 8. Cell-Based Internalization Assay of Anti-CLDN18.2 Antibodies

The humanized anti-CLDN18.2 antibodies of the disclosure were conjugated with DT3C, a recombinant protein composed of diphtheria toxin (DT) without receptor-binding domain and the C1, C2 and C3 domains of Streptococcus protein G (3C), that can reduce cell viability when internalized into cells with the antibodies, and tested for their internalization efficiency in a cell-based internalization assay.

Briefly, the recombinant protein termed DT3C was prepared in house with SEQ ID NO: 20. Then, 1.5×10⁴ 293T-CLDN18.2 cells in 100 μL DMEM medium (Cat #10566-016, Gibco) supplemented with 10% FBS were plated onto each well of 96 well-plates (Cat #3903, Corning). Meanwhile, the anti-CLDN18.2 antibodies of the disclosure or controls, 0.6 nM in DMEM medium with 10% FBS, were mixed with DT3C proteins, 1.32 nM in DMEM medium with 10% FBS, at 1:1 volume ratio, and incubated at room temperature for 30 minutes. Then, 100 si of serially diluted antibody/DT3C mixtures (4-fold serial dilution in the culture medium) were added to the cell plates of 293T-CLDN18.2 cells, and incubated in a CO₂ incubator at 37° C. for 72 hours. The plates were added with Cell Titer Glo reagent (Cat #DD1101-02, Vazyme Biotech Co., Ltd) and incubated for 10 minutes. The cell culture plates were then analyzed by Tecan infinite 200Pro plate-reader. Data were analyzed using Graphpad prism and IC₅₀ values were reported as the antibody concentrations that achieved 50% of maximal inhibition on cell viability.

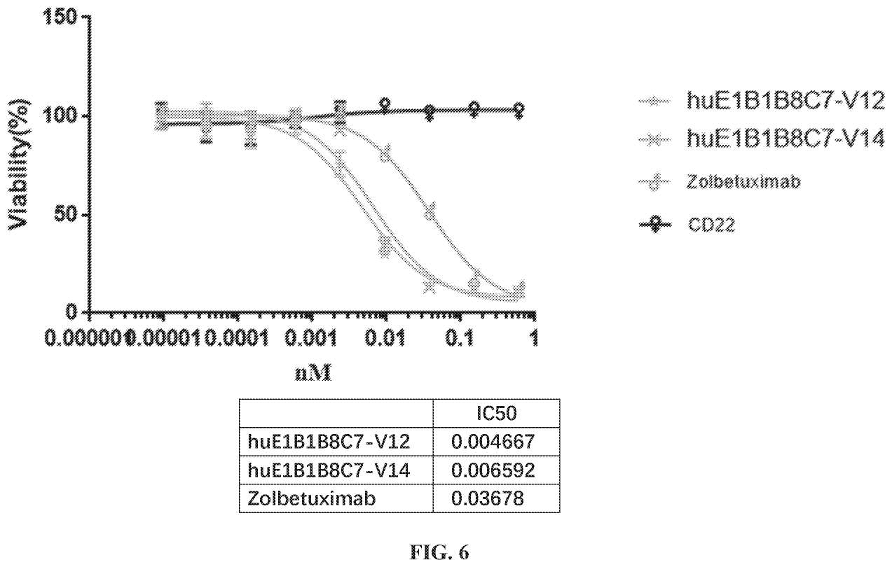

The result was shown in FIG. 6.

FIG. 6 showed that the antibody-DT3C conjugates of the disclosure were internalized at higher rates compared to the Zolbetuximab-DT3C conjugate. Specifically, huE1B1B8C7-V12-DT3C and huE1B1B8C7-V14-DT3C conjugates were more efficiently internalized by the target cells, causing target cell death in a more efficient manner.

### Example 9. Cell-Based Antibody-Dependent Cellular Cytotoxicity (ADCC) Assay

The ADCCs induced by anti-CLDN18.2 humanized antibodies huE1B1B8C7-V12 and huE1B1B8C7-V14 against target cells were measured using a luciferase detection system (Bio-Lite™ Luciferase Assay system, Cat #DD1201-02, Vazyme Biotech Co., Ltd). Jurkat-NFAT-CD16a stable cell line, stably expressing human CD16a on the cell membrane, was in house prepared by transfecting Jurkat cells with pGL4.30 plasmids (Cat #pGL4.30[luc2P/NFAT-RE/Hygro], Promega) containing an NFAT response element (NFAT-RE) that drives transcription of the luciferase reporter gene luc2P (Photinus pyralis) and pUNO1-hFCGR3Ac plasmids (Cat #pUNO1-hFCGR3Ac, Invivogene), following the manual of lipofectamine 3000 transfection reagent (Thermo Fisher), and served as effector cells in the ADCC assay. BAF3-CLDN18.2, KATO III (ATCC #HTB-103) and 293T-CLDN18.2 cells were chosen as the target cells.

Specifically, 1.25×10⁴ BAF3-CLDN18.2 cells in 100 μL RPMI1640 medium (Cat #11879-093. Gibco) supplemented with 10% FBS (Cat #10099-141, Gibco) were seeded onto 96-well plates, and incubated with 50 μl serially diluted anti-CLDN18.2 humanized antibodies (starting at 333.33 nM, with a 6-fold serial dilution in RPMI1640 medium with 10% FBS) for 1 hour in a CO₂ incubator at 37° C. Then, the plates were added with 7.5×10⁴ effector cells per well in 50 μL RPMI1640 medium supplemented with 10% FBS at an E/T ratio of 6:1, and incubated for 6 hours at 37° C. in a humidified atmosphere casing with 5% CO₂. Then, 100 μl supernatant was discarded per well. The plates were added and incubated with Luciferase detection Reagent (50 μL/well) for 10 minutes, and analyzed by Tecan infinite 200Pro plate-reader. Luminescence signals were analyzed using Graphpad prism and EC₅₀ values were reported.

For the assay involving KATO III target cells, DMEM medium was used instead of RPMI1640 medium. For the assay with 293T-CLDN18.2 target cells, DMEM medium was used instead of RPMI1640 medium, and the anti-CLDN18.2 humanized antibodies were diluted starting from 10 nM, with a 4-fold serial dilution in DMEM medium with 10% FBS.

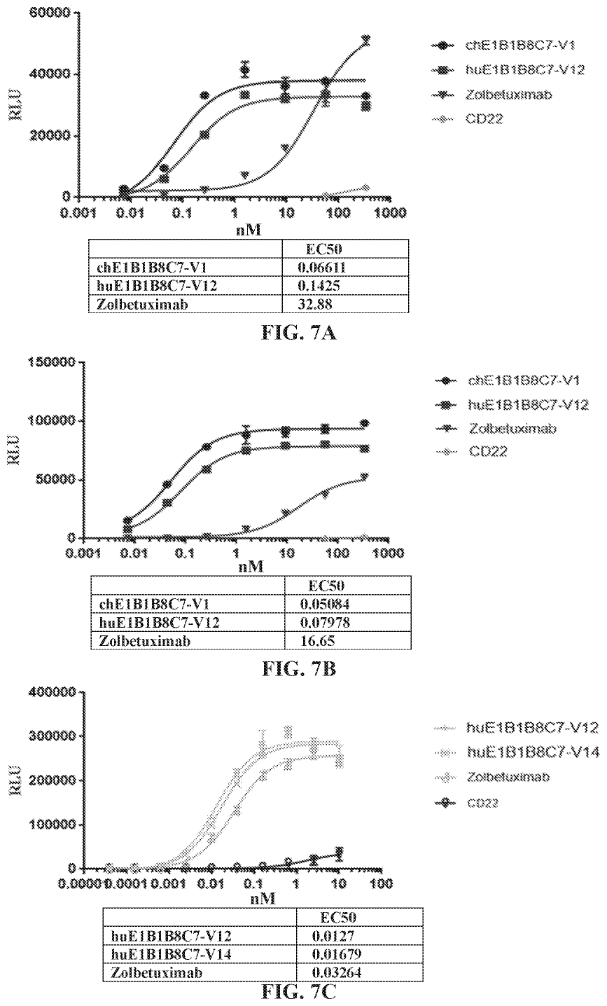

The results were shown in FIGS. 7A-7C.

As shown in FIGS. 7A-7C, huE1B1B8C7-V12 and huE1B1B8C7-V14 antibodies induced potent ADCCs against target cells, including BAF3-CLDN18.2, KATO III and 293T-CLDN18.2 cells, by Jurkat-NFAT-CD16a cells in a dose dependent manner. In specific, the huE1B1B8C7-V12 and huE1B1B8C7-V14 antibodies induced higher ADCCs than the benchmark.

### Example 10. Cytotoxicity of HuE1B1B8C7-V12-Toxin Conjugates

293T-CLDN18.2 cells and BAF3-CLDN18.2 cells respectively expressing high and middle levels of CLDN18.2, and BAF3-CLDN18.1 cells expressing CLDN18.1, as generated in Example 1, were used to evaluate the cytotoxicity of toxin (DT3C or MC-GGFG-Dxd) conjugated huE1B1B8C7-V12. In addition to the DT3C protein, the antibody was also linked to the MC-GGFG-Dxd, the linker-payload used in the HER2 targeting ADC trastuzumab deruxtecan. The huE1B1B8C7-V12-MC-GGFG-Dxd and Zolbetuximab-MC-GGFG-Dxd conjugates were prepared by a CDMO company MabPlex (China) with drug-to-antibody ratio (DAR) around 8.0.

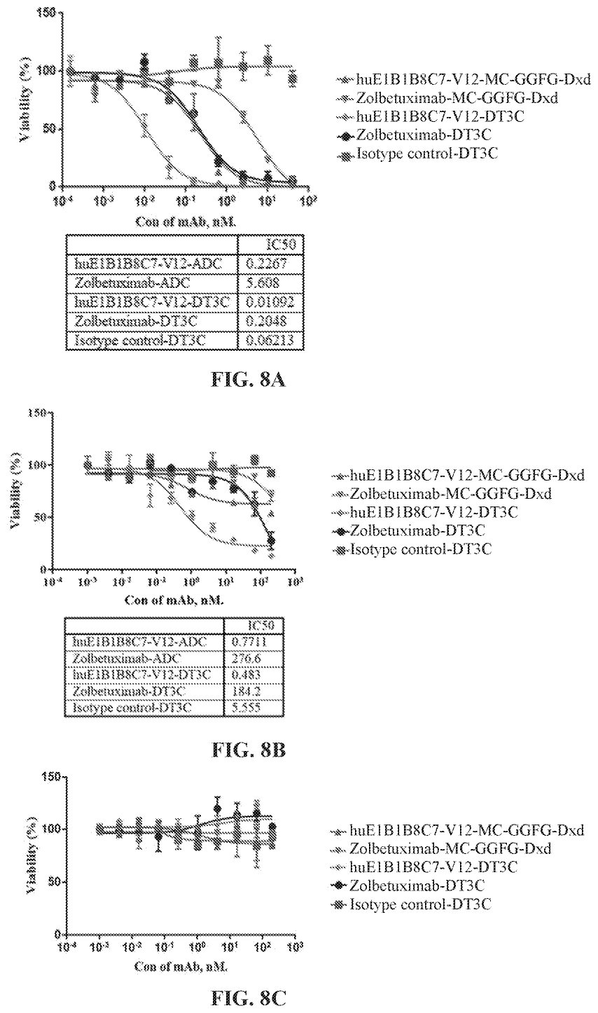

Briefly, 500 293T-CLDN18.2 cells per well in 100 μL DMEM with 10% FBS were added to 96-well cell culture plates. On the next day, anti-CLDN18.2 antibodies or the isotype control (40 nM in DMEM medium), were mixed with DT3C-his (SEQ ID NO: 20, 88 nM in DMEM medium) at 1:1 volume ratio, and incubated at room temperature for 30 minutes to form antibody-DT3C conjugates. Then, 50 μL of the serially diluted antibody-MC-GGFG-Dxd or antibody-DT3C conjugates (4-fold serially diluted in DMEM, starting from 40 nM) were added to the cell plates, and incubated in a 5% CO₂ incubator at 37° C. for 6 days. After incubation, the plates were added and incubated with Cell Counting-Lite 2.0 reagent (50 μL per well) for 5 minutes, and analyzed in Tecan infinite 200Pro plate-reader. Luminescence signals were analyzed using Graphpad prism and IC₅₀ values were reported. The results were shown in FIG. 8A.

For BAF3-CLDN18.2 and BAF3-CLDN18.1 cells, 1500 cells per well in 100 μL RPMI1640 with 10% FBS were respectively added to 96-well cell culture plates. The anti-CLDN18.2 antibodies or isotype control (200 nM in RPMI1640) were mixed with DT3C-his (440 nM in RPMI1640) at 1:1 volume ratio, and incubated at room temperature for 30 minutes to form antibody-DT3C conjugates. Then, 50 μL of the serially diluted antibody-MC-GGFG-Dxd) or antibody-DT3C conjugates (3-fold serially diluted in RPMI1640, starting from 200 nM) were added to the cell plates, and incubated in a 5% CO₂ incubator at 37° C. for 6 days. The results were shown in FIGS. 81B-8C.

As shown in FIGS. 8A-8, both huE1B1B8C7-V12 and Zolbetuximab, when conjugated with toxins (both DT3C and MC-GGCFG-Dxd), showed cytotoxicity against 293T-CLDN18.2 cells and BAF3-CLDN18.2 cells. In particular, the cytotoxicity of huE1B1B8C7-V12-toxin conjugates against 293T-CLDN18.2 cells were 20 to 30-fold higher than that of Zolbetuximab-toxin conjugates, indicating the higher internalization activity of huE1B1B8C7-V12. Similar results were observed in BAF3-CLDN18.2 cells, that is, when conjugated with either MC-GGFG-Dxd or DT3C, huE1B1B8C7-V12 showed significantly higher cytotoxicity than Zolbetuximab. According to FIG. 8C, none of the ADCs showed any non-specific cytotoxicity against BAF3-CLDN18.1 cells.

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

The important sequences in the present application are set forth below.

Description/
Sequence/SEQ ID NO.
VH CDR1 of mouse, chimeric and humanized E1B1B8C7 antibodies
NYWX1N (SEQ ID NO: 1) X1 = V
NYWVN
VH CDR2 of mouse, chimeric and humanized E1B1B8C7 antibodies
MX1HPSGSEX2RLNQX3FX4D (SEQ ID NO: 2) X1 = V, X2 = T, X3 = Q, X4 = K
MVHPSGSETRLNQQFKD
VH CDR3 of mouse, chimeric and humanized E1B1B8C7 antibodies
VWSGNAFDY (SEQ ID NO: 3)
VL CDR1 of mouse E1B1B8C7
X1SSQSLLX2X3GNQX4NYLT (SEQ ID NO: 4) X1 = L, X2 = N, X3 = S, X4 = R
LSSQSLLNSGNQRNYLT
VL CDR1 of chE1B1B8C7-V1 and humanized E1B1B8C7 antibodies
X1SSQSLLX2X3GNQX4NYLT (SEQ ID NO: 4) X1 = L, X2 = A, X3 = S, X4 = R
LSSQSLLASGNQRNYLT
VL CDR1 of chE1B1B8C7-V2
X1SSQSLLX2X3GNQX4NYLT (SEQ ID NO: 4) X1 = L, X2 = N, X3 = A, X4 = R
LSSQSLLNAGNQRNYLT
VL CDR1 of chE1B1B8C7-V3
X1SSQSLLX2X3GNQX4NYLT (SEQ ID NO: 4) X1 = L, X2 = N, X3 = E, X4 = R
LSSQSLLNEGNQRNYLT
VL CDR2 of mouse, chimeric and humanized E1B1B8C7 antibodies
WASTRDS (SEQ ID NO: 5)
VL CDR3 of mouse, chimeric and humanized E1B1B8C7 antibodies
QNGFSFPFT (SEQ ID NO: 6)
VH of mouse E1B1B8C7 antibody,
QVQLQQPGAELVRPGTSVKLSCKASGYSFTNYWVNWVKQRPGQGLEWIGMVHPSGSETR
LNQQFKDKATLTVDKSSNTAYMQLNRPTSEDSAVYYCARVWSGNAFDYWGQGTTLIVSS
(SEQ ID NO: 7)
CAGGTCCAGCTGCAGCAGCCTGGGGCTGAGCTGGTGAGGCCTGGAACTTCAGTGAAGC
TGTCCTGCAAGGCTTCTGGTTATTCCTTCACCAACTACTGGGTGAACTGGGTGAAGCAG
AGGCCTGGACAAGGCCTTGAGTGGATTGGCATGGTTCATCCTTCCGGTAGCGAAACTA
GGTTAAATCAGCAGTTCAAGGACAAGGCCACATTGACTGTAGACAAATCGTCCAATAC
AGCCTATATGCAACTCAATAGGCCGACATCTGAAGACTCTGCGGTCTATTACTGTGCA
AGGGTCTGGTCCGGTAACGCCTTTGACTACTGGGGCCAAGGCACCACTCTCATAGTCT
CCTCA (SEQ ID NO: 21)
VL of mouse E1B1B8C7 antibody
DIVMTQSPSSLTVTAGEKVTLNCLSSQSLLX1X2GNQRNYLTWYQQKPGQPPKLLIYWAST
RDSGVPDRFTGGGSGVDFTLTIYRVQAEDLAFYYCQNGFSFPFTFGSGTKLEMK (SEQ ID
NO: 8) X1 = N, X2 = S
DIVMTQSPSSLTVTAGEKVTLNCLSSQSLLNSGNQRNYLTWYQQKPGQPPKLLIYWASTR
DSGVPDRFTGGGSGVDFTLTIYRVQAEDLAFYYCQNGFSFPFTFGSGTKLEMK
GACATTGTGATGACGCAGTCTCCATCCTCCCTGACTGTGACAGCAGGAGAGAAGGTCA
CTTTGAACTGCCTGTCCAGTCAGAGTCTGTTAAACAGTGGAAATCAAAGAAACTACTT
GACCTGGTACCAGCAGAAACCAGGGCAGCCTCCTAAATTGTTGATCTATTGGGCATCC
ACTAGGGATTCTGGGGTCCCTGATCGCTTCACAGGCGGTGGATCTGGAGTAGATTTCA
CTCTCACCATCTACAGGGTGCAGGCTGAAGACCTGGCATTTTATTACTGTCAGAATGGT
TTCAGTTTTCCATTCACGTTCGGCTCGGGGACAAAGTTGGAAATGAAA (SEQ ID NO: 22)
VH of chE1B1B8C7-V1, chE1B1B8C7-V2 and chE1B1B8C7-V3
SEQ ID NO: 7
CAAGTGCAGCTGCAGCAGCCTGGCGCCGAGCTGGTGAGACCTGGCACAAGCGTGAAG
CTGAGCTGCAAGGCTAGCGGCTACAGCTTCACCAACTACTGGGTGAACTGGGTGAAGC
AGAGACCTGGCCAAGGCCTGGAGTGGATCGGCATGGTGCACCCTAGCGGCAGCGAGA
CAAGACTGAATCAGCAGTTCAAGGACAAGGCCACCCTGACCGTGGACAAGAGCAGCA
ACACCGCCTACATGCAGCTGAACAGACCTACAAGCGAGGACAGCGCCGTGTACTACTG
CGCTAGAGTGTGGAGCGGCAACGCCTTCGACTACTGGGGCCAAGGCACCACCCTGATC
GTGAGCAGC (SEQ ID NO: 23)
VL of chE1B1B8C7-V1
SEQ ID NO: 8, X1 = A, X2 = S
DIVMTQSPSSLTVTAGEKVTLNCLSSQSLLASGNQRNYLTWYQQKPGQPPKLLIYWASTR
DSGVPDRFTGGGSGVDFTLTIYRVQAEDLAFYYCQNGESFPFTFGSGTKLEMK
GACATCGTGATGACACAGAGCCCTAGCAGCCTGACCGTGACCGCCGGCGAGAAGGTG
ACCCTGAACTGCCTGAGCAGCCAAAGCCTGCTGR1MCR2VBGGCAATCAGAGAAACTA
CCTGACCTGGTATCAGCAGAAGCCTGGACAGCCTCCTAAGCTGCTGATCTACTGGGCT
AGCACAAGAGACAGCGGCGTGCCTGACAGATTCACCGGCGGAGGCAGCGGCGTGGAC
TTCACCCTGACCATCTACAGAGTGCAAGCCGAGGACCTGGCCTTCTACTACTGTCAGA
ACGGCTTCAGCTTCCCTTTCACCTTCGGCAGCGGCACCAAGCTGGAGATGAAG (SEQ ID
NO: 24) R1 = G, M = C, R2 = A, V = G, B = C
GACATCGTGATGACACAGAGCCCTAGCAGCCTGACCGTGACCGCCGGCGAGAAGGTG
ACCCTGAACTGCCTGAGCAGCCAAAGCCTGCTGGCCAGCGGCAATCAGAGAAACTAC
CTGACCTGGTATCAGCAGAAGCCTGGACAGCCTCCTAAGCTGCTGATCTACTGGGCTA
GCACAAGAGACAGCGGCGTGCCTGACAGATTCACCGGCGGAGGCAGCGGCGTGGACT
TCACCCTGACCATCTACAGAGTGCAAGCCGAGGACCTGGCCTTCTACTACTGTCAGAA
CGGCTTCAGCTTCCCTTTCACCTTCGGCAGCGGCACCAAGCTGGAGATGAAG
VL of chE1B1B8C7-V2
SEQ ID NO: 8, X1 = N, X2 = A
DIVMTQSPSSLTVTAGEKVTLNCLSSQSLLNAGNQRNYLTWYQQKPGQPPKLLIYWASTR
DSGVPDRFTGGGSGVDFTLTIYRVQAEDLAFYYCQNGFSFPFTFGSGTKLEMK
SEQ ID NO: 24, R1 = A, M = A, R2 = G, V = C, B = T
GACATCGTGATGACACAGAGCCCTAGCAGCCTGACCGTGACCGCCGGCGAGAAGGTG
ACCCTGAACTGCCTGAGCAGCCAAAGCCTGCTGAACGCTGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCACCGGCGGAGGCAGCGGCGTGGACTT
CACCCTGACCATCTACAGAGTGCAAGCCGAGGACCTGGCCTTCTACTACTGTCAGAAC
GGCTTCAGCTTCCCTTTCACCTTCGGCAGCGGCACCAAGCTGGAGATGAAG
VL of chE1B1B8C7-V3
SEQ ID NO: 8, X1 = N, X2 = E
DIVMTQSPSSLTVTAGEKVTLNCLSSQSLLNEGNQRNYLTWYQQKPGQPPKLLIYWASTR
DSGVPDRFTGGGSGVDFTLTIYRVQAEDLAFYYCQNGFSFPFTFGSGTKLEMK
SEQ ID NO: 24, R1 = A, M = A, R2 = G, V = A, B = G
GACATCGTGATGACACAGAGCCCTAGCAGCCTGACCGTGACCGCCGGCGAGAAGGTG
ACCCTGAACTGCCTGAGCAGCCAAAGCCTGCTGAACGAGGGCAATCAGAGAAACTAC
CTGACCTGGTATCAGCAGAAGCCTGGACAGCCTCCTAAGCTGCTGATCTACTGGGCTA
GCACAAGAGACAGCGGCGTGCCTGACAGATTCACCGGCGGAGGCAGCGGCGTGGACT
TCACCCTGACCATCTACAGAGTGCAAGCCGAGGACCTGGCCTTCTACTACTGTCAGAA
CGGCTTCAGCTTCCCTTTCACCTTCGGCAGCGGCACCAAGCTGGAGATGAAG
VH of huE1B1B8C7-V1, huE1B1B8C7-V9, huE1B1B8C7-V12 and huE1B1B&C7-V15
QVQLVQSGAEVKKPGASVKVSCKASGYX1FTNYWVNWVRQAPGQGLEWX2GMVHPSGS
ETRLNQQFKDKATLTVDX3STSTX4YMELSSLRSEDTAVYYCARVWSGNAFDYWGQGTL
VTVSS (SEQ ID NO: 9) X1 = S, X2 = 1, X3 = K, X4 = A
QVQLVQSGAEVKKPGASVKVSCKASGYSFTNYWVNWVRQAPGQGLEWIGMVHPSGSET
RLNQQFKDKATLTVDKSTSTAYMELSSLRSEDTAVYYCARVWSGNAFDYWGQGTLVTVS
S
CAAGTGCAGCTGGTGCAGAGCGGCGCCGAGGTGAAGAAGCCTGGCGCTAGCGTGAAG
GTGAGCTGCAAGGCTAGCGGCTACASCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCCTGACCGTGGACAMRAGCACAA
GCACCGYSTACATGGAGCTGAGCAGCCTGAGAAGCGAGGACACCGCCGTGTACTACTG
CGCTAGAGTGTGGAGCGGCAACGCCTTCGACTACTGGGGCCAAGGCACCCTGGTGACC
GTGAGCAGC (SEQ ID NO.: 25) S = G, M = A, R = G, Y = C, S = C
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCCTGACCGTGGACAAGAGCACAA
GCACCGCCTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V2, huE1B1B8C7-V10, huE1B1B8C7-V13 and huE1B1B8C7-V16
SEQ ID NO: 9, X1 = S, X2 = I, X3 = T, X4 = V
QVQLVQSGAEVKKPGASVKVSCKASGYSFTNYWVNWVRQAPGQGLEWIGMVHPSGSET
RLNQQFKDKATLTVDTSTSTVYMELSSLRSEDTAVYYCARVWSGNAFDYWGQGTLVTVS
S
SEQ ID NO.: 25, S = G, M = C, R = A, Y = T, S = G
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCCTGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V3, huE1B1B8C7-V11, huE1B1B8C7-V14 and huE1B1B8C7-V17
SEQ ID NO: 9, X1 = T, X2 = I, X3 = T, X4= V
QVQLVQSGAEVKKPGASVKVSCKASGYTFTNYWVNWVRQAPGQGLEWIGMVHPSGSET
RLNQQFKDKATLTVDTSTSTVYMELSSLRSEDTAVYYCARVWSGNAFDYWGQGTLVTVS
S
SEQ ID NO.: 25, S = C, M = C, R = A, Y = T, S = G
CAAGTGCAGCTGGTGCAGAGCGGCGCCGAGGTGAAGAAGCCTGGCGCTAGCGTGAAG
GTGAGCTGCAAGGCTAGCGGCTACACCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCCTGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V4
SEQ ID NO: 9, X1 = S, X2 = M, X3 = T, X4 = V
QVQLVQSGAEVKKPGASVKVSCKASGYSFTNYWVNWVRQAPGQGLEWMGMVHPSGSE
TRLNQQFKDKATLTVDTSTSTVYMELSSLRSEDTAVYYCARVWSGNAFDYWGQGTLVTV
SS
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATS1GGCATGGTGCACCCTAGCGGCAGCGAGA
CAAGACTGAATCAGCAGTTCAAGGACAR1R2GYS2ACCCTGACCGTGGACACAAGCAC
AAGCACCGTGTACATGGAGCTGAGCAGCCTGAGAAGCGAGGACACCGCCGTGTACTA
CTGCGCTAGAGTGTGGAGCGGCAACGCCTTCGACTACTGGGGCCAAGGCACCCTGGTG
ACCGTGAGCAGC (SEQ ID NO: 26) S1 = G, R1 = A, R2 = G, Y = C, S2 = C
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATGGGCATGGTGCACCCTAGCGGCAGCGAGA
CAAGACTGAATCAGCAGTTCAAGGACAAGGCCACCCTGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V5
QVQLVQSGAEVKKPGASVKVSCKASGYSFTNYWVNWVRQAPGQGLEWIGMVHPSGSET
RLNQQFKDX1X2TX3TX4DTSTSTVYMELSSLRSEDTAVYYCARVWSGNAFDYWGQGTL
VTVSS (SEQ ID NO: 10) X1 = R, X2 = A, X3 = L, X4 = V
QVQLVQSGAEVKKPGASVKVSCKASGYSFTNYWVNWVRQAPGQGLEWIGMVHPSGSET
RLNQQFKDRATLTVDTSTSTVYMELSSLRSEDTAVYYCARVWSGNAFDYWGQGTLVTVS
S
SEQ ID NO: 26, S1 = C, R1 = G, R2 = A, Y = C, S2 = C
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAGAGCCACCCTGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V6
SEQ ID NO: 10, X1 = K, X2 = V, X3 = L, X4 = V
QVQLVQSGAEVKKPGASVKVSCKASGYSFTNYWVNWVRQAPGQGLEWIGMVHPSGSET
RLNQQFKDKVTLTVDTSTSTVYMELSSLRSEDTAVYYCARVWSGNAFDYWGQGTLVTVS
S
SEQ ID NO: 26, S1 = C, R1 = A, R2 = G, Y = T, S2 = G
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGTGACCCTGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V7
SEQ ID NO: 10, X1 = K, X2 = A, X3 = M, X4 = V
QVQLVQSGAEVKKPGASVKVSCKASGYSFTNYWVNWVRQAPGQGLEWIGMVHPSGSET
RLNQQFKDKATMTVDTSTSTVYMELSSLRSEDTAVYYCARVWSGNAFDYWGQGTLVTV
SS
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCMTGACCR1KR2GACACAAGCAC
AAGCACCGTGTACATGGAGCTGAGCAGCCTGAGAAGCGAGGACACCGCCGTGTACTA
CTGCGCTAGAGTGTGGAGCGGCAACGCCTTCGACTACTGGGGCCAAGGCACCCTGGTG
ACCGTGAGCAGC (SEQ ID NO: 27) M = A, R1 = G, K = T, R2 = G
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCATGACCGTGGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VH of huE1B1B8C7-V8
SEQ ID NO: 10, X1 = K, X2 = A, X3 = L, X4 = R
QVQLVQSGAEVKKPGASVKVSCKASGYSFTNYWVNWVRQAPGQGLEWIGMVHPSGSET
RLNQQFKDKATLTRDTSTSTVYMELSSLRSEDTAVYYCARVWSGNAFDYWGQGTLVTVS
S
SEQ ID NO: 27, M = C, R1 = A, K = G, R2 = A
CAAGTGCAGCTGGTGCAGAGCGGCGCCGAGGTGAAGAAGCCTGGCGCTAGCGTGAAG
GTGAGCTGCAAGGCTAGCGGCTACAGCTTCACCAACTACTGGGTGAACTGGGTGAGAC
AAGCCCCTGGCCAAGGCCTGGAGTGGATCGGCATGGTGCACCCTAGCGGCAGCGAGA
CAAGACTGAATCAGCAGTTCAAGGACAAGGCCACCCTGACCAGAGACACAAGCACAA
GCACCGTGTACATGGAGCTGAGCAGCCTGAGAAGCGAGGACACCGCCGTGTACTACT
GCGCTAGAGTGTGGAGCGGCAACGCCTTCGACTACTGGGGCCAAGGCACCCTGGTGAC
CGTGAGCAGC
VL of huE1B1B8C7-V1-huE1B1B8C7-V8
DIVMTQSPDSLAVSLGERATINCLSSQSLLASGNQRNYLTWYQQKPGQPPKLLIYWASTRD
SGVPDRFX1GSGSGX2DFTLTISSX3QAEDVAVYYCQNGFSFPFTFGQGTKLEIK (SEQ ID
NO: 11) X1 = T, X2 = V, X3 = V
DIVMTQSPDSLAVSLGERATINCLSSQSLLASGNQRNYLTWYQQKPGQPPKLLIYWASTRD
SGVPDRFTGSGSGVDFTLTISSVQAEDVAVYYCQNGFSFPFTFGQGTKLEIK
GACATCGTGATGACACAGAGCCCTGACAGCCTGGCCGTGAGCCTGGGCGAGAGAGCC
ACCATCAACTGCCTGAGCAGCCAAAGCCTGCTGGCTAGCGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCAS1CGGCAGCGGCAGCGGCRYS2GACT
TCACCCTGACCATCAGCAGCS3TGCAAGCCGAGGACGTGGCCGTGTACTACTGTCAGA
ACGGCTTCAGCTTCCCTTTCACCTTCGGCCAAGGCACCAAGCTGGAGATCAAG (SEQ ID
No: 28) S1 = C, R = G, Y = T, S2 = G, S3 = G
GACATCGTGATGACACAGAGCCCTGACAGCCTGGCCGTGAGCCTGGGCGAGAGAGCC
ACCATCAACTGCCTGAGCAGCCAAAGCCTGCTGGCTAGCGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCACCGGCAGCGGCAGCGGCGTGGACTT
CACCCTGACCATCAGCAGCGTGCAAGCCGAGGACGTGGCCGTGTACTACTGTCAGAAC
GGCTTCAGCTTCCCTTTCACCTTCGGCCAAGGCACCAAGCTGGAGATCAAG
VL of huE1B1B8C7-V9-huE1B1B8C7-V11
SEQ ID NO: 11, X1 = S, X2 = V, X3 = V
DIVMTQSPDSLAVSLGERATINCLSSQSLLASGNQRNYLTWYQQKPGQPPKLLIYWASTRD
SGVPDRFSGSGSGVDFTLTISSVQAEDVAVYYCQNGFSFPFTFGQGTKLEIK
SEQ ID No: 28, S1 = G, R = G, Y = T, S2 = G, S3 = G
GACATCGTGATGACACAGAGCCCTGACAGCCTGGCCGTGAGCCTGGGCGAGAGAGCC
ACCATCAACTGCCTGAGCAGCCAAAGCCTGCTGGCTAGCGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCAGCGGCAGCGGCAGCGGCGTGGACTT
CACCCTGACCATCAGCAGCGTGCAAGCCGAGGACGTGGCCGTGTACTACTGTCAGAAC
GGCTTCAGCTTCCCTTTCACCTTCGGCCAAGGCACCAAGCTGGAGATCAAG
VL of huE1B1B8C7-V12-huE1B1B8C7-V14
SEQ ID NO: 11, X1 = T, X2 = T, X3 = V
DIVMTQSPDSLAVSLGERATINCLSSQSLLASGNQRNYLTWYQQKPGQPPKLLIYWASTRD
SGVPDRFTGSGSGTDFTLTISSVQAEDVAVYYCQNGFSFPFTFGQGTKLEIK
SEQ ID No: 28, S1 = C, R = A, Y = C, S2 = C, S3 = G
GACATCGTGATGACACAGAGCCCTGACAGCCTGGCCGTGAGCCTGGGCGAGAGAGCC
ACCATCAACTGCCTGAGCAGCCAAAGCCTGCTGGCTAGCGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCACCGGCAGCGGCAGCGGCACCGACTT
CACCCTGACCATCAGCAGCGTGCAAGCCGAGGACGTGGCCGTGTACTACTGTCAGAAC
GGCTTCAGCTTCCCTTTCACCTTCGGCCAAGGCACCAAGCTGGAGATCAAG
VL of huE1B1B8C7-V15-huE1B1B8C7-V17
SEQ ID NO: 11, X1 = T, X2 = V, X3 = L
DIVMTQSPDSLAVSLGERATINCLSSQSLLASGNQRNYLTWYQQKPGQPPKLLIYWASTRD
SGVPDRFTGSGSGVDFTLTISSLQAEDVAVYYCQNGFSFPFTFGQGTKLEIK
SEQ ID No: 28, S1 = C, R = G, Y = T, S2 = G, S3 = C
GACATCGTGATGACACAGAGCCCTGACAGCCTGGCCGTGAGCCTGGGCGAGAGAGCC
ACCATCAACTGCCTGAGCAGCCAAAGCCTGCTGGCTAGCGGCAATCAGAGAAACTACC
TGACCTGGTATCAGCAGAAGCCTGGACAGCCTCCTAAGCTGCTGATCTACTGGGCTAG
CACAAGAGACAGCGGCGTGCCTGACAGATTCACCGGCAGCGGCAGCGGCGTGGACTT
CACCCTGACCATCAGCAGCCTGCAAGCCGAGGACGTGGCCGTGTACTACTGTCAGAAC
GGCTTCAGCTTCCCTTTCACCTTCGGCCAAGGCACCAAGCTGGAGATCAAG
VH CDR1 of mouse E1A1F4B5
NYWX1N (SEQ ID NO: 1) X1 = V
NYWVN
VH CDR2 for mouse E1A1F4B5
MX1HPSGSEX2RLNQX3FX4D (SEQ ID NO: 2) X1 = I, X2 = T, X3 = R, X4 = R
MIHPSGSETRLNQRFRD
VH CDR3 of mouse E1A1F4B5
VWSGNAFDY (SEQ ID NO: 3)
VL CDR1 of mouse E1A1F4B5
X1SSQSLLX2X3GNQX4NYLT (SEQ ID NO: 4) X1 = M, X2 = N, X3 = S, X4 = K
MSSQSLLNSGNQKNYLT
VL CDR2 of mouse E1A1F4B5
WASTRDS (SEQ ID NO: 5)
VL CDR3 of mouse E1A1F4B5
QNGFSFPFT (SEQ ID NO: 6)
VH of mouse E1A1F4B5
QVQLQQPGAELVRPGTSVKLSCKASGYSFTNYWVNWVKQRPGQGLEWIGMIHPSGSETR
LNQRFRDKTTLTVDRSSNTAYMQLNRPTSEDSAVYYCARVWSGNAFDYWGQGTTLIVSS
(SEQ ID NO: 12)
CAGGTCCAGCTGCAGCAGCCTGGGGCTGAGCTGGTGAGGCCTGGAACTTCAGTGAAGC
TGTCCTGCAAGGCTTCTGGTTATTCCTTCACCAACTACTGGGTGAACTGGGTGAAGCAG
AGGCCTGGACAAGGCCTTGAGTGGATTGGCATGATTCATCCTTCCGGTAGCGAAACTA
GATTAAATCAGCGGTTCAGGGACAAGACCACATTGACTGTAGACAGATCGTCCAATAC
AGCCTATATGCAACTCAATAGGCCGACATCTGAAGACTCTGCGGTCTATTACTGTGCA
AGGGTCTGGTCCGGTAACGCCTTTGACTACTGGGGCCAAGGCACCACTCTCATAGTCT
CCTCA (SEQ ID NO: 29)
VL of mouse E1A1F4B5
DIVMTQSPSSLTVTAGEKVTLNCMSSQSLLNSGNQKNYLTWYQQKPGQPPKLLIYWASTR
DSGVPDRFTGGGSGVDFTLTIYRVQAEDLAFYFCQNGFSFPFTFGSGTKLEMK (SEQ ID
NO: 13)
GACATTGTGATGACGCAGTCTCCATCCTCCCTGACTGTGACAGCAGGAGAGAAGGTCA
CTTTGAACTGCATGTCCAGTCAGAGTCTGTTAAACAGTGGAAATCAAAAAAACTACTT
GACCTGGTACCAGCAGAAACCAGGGCAGCCTCCTAAATTGTTGATCTATTGGGCATCC
ACTAGGGATTCTGGGGTCCCTGATCGCTTCACAGGCGGTGGATCTGGAGTAGATTTCA
CTCTCACCATCTACAGGGTGCAGGCTGAAGACCTGGCATTTTATTTCTGTCAGAATGGT
TTCAGTTTTCCATTCACGTTCGGCTCGGGGACAAAGTTGGAAATGAAA (SEQ ID NO: 30)
VH CDR1 for mouse E1H4C3E5
NYWX1N (SEQ ID NO: 1) X1 = M
NYWMN
VH CDR2 for mouse E1H4C3E5
MX1HPSGSEX2RLNQX3FX4D (SEQ ID NO: 2) X1 = I, X2 = S, X3 = K, X4 = K
MIHPSGSESRLNQKFKD
VH CDR3 of mouse E1H4C3E5
VWSGNAFDY (SEQ ID NO: 3)
VL CDR1 of mouse E1H4C3E5
X1SSQSLLX2X3GNQX4NYLT (SEQ ID NO: 4) X1 = M, X2 = N, X3 = S, X4 = K
MSSQSLLNSGNQKNYLT
VL CDR2 of mouse E1H4C3E5
WASTRDS (SEQ ID NO: 5)
VL CDR3 of mouse E1H4C3E5
QNGFSFPFT (SEQ ID NO: 6)
VH of mouse E1H4C3E5
QVQLQQPGAELVRPGTSVKLSCKASGYSFTNYWMNWVKQRPGQGLEWIGMIHPSGSESR
LNQKFKDKATLTVDKSSNTAYMQLNRPTSADSAVYYCARVWSGNAFDYWGQGTTLIVSS
(SEQ ID NO: 14)
CAGGTCCAGCTGCAGCAGCCTGGGGCTGAGCTGGTGAGGCCTGGAACTTCAGTGAAGC
TGTCCTGCAAGGCTTCTGGTTATTCCTTCACCAACTACTGGATGAACTGGGTGAAACAG
AGGCCTGGACAAGGCCTTGAGTGGATTGGCATGATTCATCCTTCCGGTAGCGAAAGTA
GGTTAAATCAGAAGTTCAAGGACAAGGCCACATTGACTGTAGACAAATCGTCCAATAC
AGCCTATATGCAACTCAATAGGCCGACATCTGCAGACTCTGCGGTCTATTACTGTGCA
AGGGTCTGGTCCGGTAACGCCTTTGATTACTGGGGCCAAGGCACCACTCTCATAGTCTC
CTCA (SEQ ID NO: 31)
VL of mouse E1H4C3E5
DIVMTQSPSSLTVTAGEKVTLSCMSSQSLLNSGNQKNYLTWYQQKPGQPPKLLIFWASTR
DSGVPDRFTGGGSGTLFTLTIYRVQAEDLAFYYCQNGFSFPFTFGSGTKLEMR (SEQ ID
NO: 15)
GACATTGTGATGACACAGTCTCCATCCTCCCTGACTGTGACAGCAGGGGAGAAGGTCA
CTTTGAGCTGCATGTCCAGTCAGAGTCTGTTAAACAGTGGAAATCAAAAAAACTACTT
GACCTGGTACCAGCAGAAACCAGGGCAGCCTCCTAAATTGTTGATCTTTTGGGCATCC
ACTAGGGATTCTGGGGTCCCTGATCGCTTCACAGGCGGTGGATCTGGAACACTTTTCAC
TCTCACCATCTACAGGGTGCAGGCTGAAGACCTGGCATTTTATTACTGTCAGAATGGTT
TCAGTTTTCCATTCACGTTCGGCTCGGGGACAAAGTTGGAAATGAGA (SEQ ID NO: 32)
Heavy chain constant region for chimeric and humanized antibodies
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGK (SEQ ID NO: 16)
Light chain constant region for chimeric and humanized antibodies
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID NO: 17)
Heavy chain of Zolbetuximab
QVQLQQPGAELVRPGASVKLSCKASGYTFTSYWINWVKQRPGQGLEWIGNIYPSDSYTNY
NQKFKDKATLTVDKSSSTAYMQLSSPTSEDSAVYYCTRSWRGNSFDYWGQGTTLTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNV
FSCSVMHEALHNHYTQKSLSLSPGK (SEQ ID NO: 18)
Light chain of Zolbetuximab
DIVMTQSPSSLTVTAGEKVTMSCKSSQSLLNSGNQKNYLTWYQQKPGQPPKLLIYWASTR
ESGVPDRFTGSGSGTDFTLTISSVQAEDLAVYYCQNDYSYPFTFGSGTKLEIKRTVAAPSVF
IFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC (SEQ ID NO: 19)
DT3C protein
MGADDVVDSSKSFVMENFSSYHGTKPGYVDSIQKGIQKPKSGTQGNYDDDWKGFYSTDN
KYDAAGYSVDNENPLSGKAGGVVKVTYPGLTKVLALKVDNAETIKKELGLSLTEPLMEQ
VGTEEFIKRFGDGASRVVLSLPFAEGSSSVEYINNWEQAKALSVELEINFETRGKRGQDAM
YEYMAQACAGNRVRRSVGSSLSCINLDWDVIRDKTKTKIESLKEHGPIKNKMSESPNKTVS
EEKAKQYLEEFHQTALEHPELSELKTVTGTNPVFAGANYAAWAVNVAQVIDSETADNLE
KTTAALSILPGIGSVMGIADGAVHHNTEEIVAQSIALSSLMVAQAIPLVGELVDIGFAAYNF
VESIINLFQVVHNSYNRPAYSPGHKHIDEILAALPKTDTYKLILNGKTLKGETTTEAVDAAT
AEKVFKQYANDNGVDGEWTYDDATKTFTVTEKPEVIDASELTPAVTTYKLVINGKTLKGE
TTTEAVDAATAEKVFKQYANDNGVDGEWTYDDATKTFTVTEKPEVIDASELTPAVTTYK
LVINGKTLKGETTTKAVDAETAEKAFKQYANDNGVDGVWTYDDATKTFTVTELEHHHH
HH (SEQ ID NO: 20)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated monoclonal antibody, or an antigen-binding portion thereof, capable of binding to CLDN18.2, comprising (i) a heavy chain variable region comprising a VH CDR1 region, a VH CDR2 region and a VH CDR3 region, and (ii) a light chain variable region comprising a VL CDR1 region, a VL CDR2 region and a VL CDR3 region, wherein
(1) the VH CDR1 region comprises the amino acid sequence NYWX1N (SEQ ID NO: 1), wherein X1 is V,
the VH CDR2 region comprises the amino acid sequence MX1HPSGSEX2RLNQX3FX4D (SEQ ID NO: 2), wherein X1, X2, X3 and X4 are V, T, Q and K, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are L, A, S and R, respectively,
the VL CDR2 comprises the amino acid sequence WASTRDS (SEQ ID NO: 5),
the VL CDR3 comprises the amino acid sequence QNGFSFPFT (SEQ ID NO: 6);
(2) the VH CDR1 region comprises the amino acid sequence NYWX1N (SEQ ID NO: 1), wherein X1 is V,
the VH CDR2 region comprises the amino acid sequence MX1HPSGSEX2RLNQX3FX4D (SEQ ID NO: 2), wherein X1, X2, X3 and X4 are V, T, Q and K, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are L, N, S and R, respectively,
the VL CDR2 comprises the amino acid sequence WASTRDS (SEQ ID NO: 5),
the VL CDR3 comprises the amino acid sequence QNGFSFPFT (SEQ ID NO: 6);
(3) the VH CDR1 region comprises the amino acid sequence NYWX1N (SEQ ID NO: 1), wherein X1 is V,
the VH CDR2 region comprises the amino acid sequence MX1HPSGSEX2RLNQX3FX4D (SEQ ID NO: 2), wherein X1, X2, X3 and X4 are V, T, Q and K, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are L, N, A and R, respectively,
the VL CDR2 comprises the amino acid sequence WASTRDS (SEQ ID NO: 5),
the VL CDR3 comprises the amino acid sequence QNGFSFPFT (SEQ ID NO: 6);
(4) the VH CDR1 region comprises the amino acid sequence NYWX1N (SEQ ID NO: 1), wherein X1 is V,
the VH CDR2 region comprises the amino acid sequence MX1HPSGSEX2RLNQX3FX4D (SEQ ID NO: 2), wherein X1, X2, X3 and X4 are V, T, Q and K, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are L, N, E and R, respectively,
the VL CDR2 comprises the amino acid sequence WASTRDS (SEQ ID NO: 5),
the VL CDR3 comprises the amino acid sequence QNGFSFPFT (SEQ ID NO: 6);
(5) the VH CDR1 region comprises the amino acid sequence NYWX1N (SEQ ID NO: 1), wherein X1 is V,
the VH CDR2 region comprises the amino acid sequence MX1HPSGSEX2RLNQX3FX4D (SEQ ID NO: 2), wherein X1, X2, X3 and X4 are I, T, R and R, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are M, N, S and K, respectively,
the VL CDR2 comprises the amino acid sequence WASTRDS (SEQ ID NO: 5),
the VL CDR3 comprises the amino acid sequence QNGFSFPFT (SEQ ID NO: 6); or
(6) the VH CDR1 region comprises the amino acid sequence NYWX1N (SEQ ID NO: 1), wherein X1 is M,
the VH CDR2 region comprises the amino acid sequence MX1HPSGSEX2RLNQX3FX4D (SEQ ID NO: 2), wherein X1, X2, X3 and X4 are I, S, K and K, respectively,
the VH CDR3 region comprises the amino acid sequence VWSGNAFDY (SEQ ID NO: 3),
the VL CDR1 comprises the amino acid sequence XISSQSLLX2X3GNQX4NYLT (SEQ ID NO: 4), wherein X1, X2, X3 and X4 are M, N, S and K, respectively,
the VL CDR2 comprises the amino acid sequence WASTRDS (SEQ ID NO: 5),
the VL CDR3 comprises the amino acid sequence QNGFSFPFT (SEQ ID NO: 6).

2. (canceled)

3. The isolated monoclonal antibody, or the antigen-binding portion thereof, of claim 1, wherein the heavy chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 7, 9, 10, 12, or 14,
wherein Xs in position 28, 48, 74 and 79 of SEQ ID NO: 9 are S, I, K and A, respectively; S, I, T and V, respectively; T, I, T and V, respectively; or S, M, T and V, respectively,
wherein Xs in position 67, 68, 70 and 72 of SEQ ID NO: 10 are R, A, L and V, respectively; K, V, L and V, respectively; K, A, M and V, respectively; or K, A, L and R, respectively.

4. The isolated monoclonal antibody, or the antigen-binding portion thereof, of claim 1, wherein the light chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 8, 11, 13, or 15,
wherein Xs in position 31 and 32 of SEQ ID NO: 8 are N and S, respectively; A and S, respectively; N and A, respectively; or N and E, respectively,
wherein Xs in position of 69, 75 and 84 of SEQ ID NO: 11 are T, V and V, respectively; S, V and V, respectively; T, T and V, respectively; or T, V and L, respectively.

5. The isolated monoclonal antibody, or an antigen-binding portion thereof, of claim 2, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to
(1) SEQ ID NOs: 9 and 11, respectively, wherein Xs in position 28, 48, 74 and 79 of SEQ ID NO: 9 are S, I, K and A, respectively, wherein Xs in position of 69, 75 and 84 of SEQ ID NO: 11 are T, T and V, respectively;
(2) SEQ ID NOs: 9 and 11, respectively, wherein Xs in position 28, 48, 74 and 79 of SEQ ID NO: 9 are S, I, K and A, respectively, wherein Xs in position of 69, 75 and 84 of SEQ ID NO: 11 are T, V and V, respectively;
(3) SEQ ID NOs: 9 and 11, respectively, wherein Xs in position 28, 48, 74 and 79 of SEQ ID NO: 9 are S, I, T and V, respectively, wherein Xs in position of 69, 75 and 84 of SEQ ID NO: 11 are T, V and V, respectively;
(4) SEQ ID NOs: 9 and 11, respectively, wherein Xs in position 28, 48, 74 and 79 of SEQ ID NO: 9 are T, I, T and V, respectively, wherein Xs in position of 69, 75 and 84 of SEQ ID NO: 11 are T, V and V, respectively;
(5) SEQ ID NOs: 9 and 11, respectively, wherein Xs in position 28, 48, 74 and 79 of SEQ ID NO: 9 are S, M, T and V, respectively, wherein Xs in position of 69, 75 and 84 of SEQ ID NO: 11 are T, V and V, respectively;
(6) SEQ ID NOs: 10 and 11, respectively, wherein Xs in position 67, 68, 70 and 72 of SEQ ID NO: 10 are R, A, L and V, respectively, wherein Xs in position of 69, 75 and 84 of SEQ ID NO: 11 are T, V and V, respectively;
(7) SEQ ID NOs: 10 and 11, respectively, wherein Xs in position 67, 68, 70 and 72 of SEQ ID NO: 10 are K, V, L and V, respectively, wherein Xs in position of 69, 75 and 84 of SEQ ID NO: 11 are T, V and V, respectively;
(8) SEQ ID NOs: 10 and 11, respectively, wherein Xs in position 67, 68, 70 and 72 of SEQ ID NO: 10 are K, A, M and V, respectively, wherein Xs in position of 69, 75 and 84 of SEQ ID NO: 11 are T, V and V, respectively;
(9) SEQ ID NOs: 10 and 11, respectively, wherein Xs in position 67, 68, 70 and 72 of SEQ ID NO: 10 are K, A, L and R, respectively, wherein Xs in position of 69, 75 and 84 of SEQ ID NO: 11 are T, V and V, respectively;
(10) SEQ ID NOs: 9 and 11, respectively, wherein Xs in position 28, 48, 74 and 79 of SEQ ID NO: 9 are S, I, K and A, respectively, wherein Xs in position of 69, 75 and 84 of SEQ ID NO: 11 are S, V and V, respectively;
(11) SEQ ID NOs: 9 and 11, respectively, wherein Xs in position 28, 48, 74 and 79 of SEQ ID NO: 9 are S, I, T and V, respectively, wherein Xs in position of 69, 75 and 84 of SEQ ID NO: 11 are S, V and V, respectively;
(12) SEQ ID NOs: 9 and 11, respectively, wherein Xs in position 28, 48, 74 and 79 of SEQ ID NO: 9 are T, I, T and V, respectively, wherein Xs in position of 69, 75 and 84 of SEQ ID NO: 11 are S, V and V, respectively;
(13) SEQ ID NOs: 9 and 11, respectively, wherein Xs in position 28, 48, 74 and 79 of SEQ ID NO: 9 are S, I, T and V, respectively, wherein Xs in position of 69, 75 and 84 of SEQ ID NO: 11 are T, T and V, respectively;
(14) SEQ ID NOs: 9 and 11, respectively, wherein Xs in position 28, 48, 74 and 79 of SEQ ID NO: 9 are T, I, T and V, respectively, wherein Xs in position of 69, 75 and 84 of SEQ ID NO: 11 are T, T and V, respectively;
(15) SEQ ID NOs: 9 and 11, respectively, wherein Xs in position 28, 48, 74 and 79 of SEQ ID NO: 9 are S, I, K and A, respectively, wherein Xs in position of 69, 75 and 84 of SEQ ID NO: 11 are T, V and L, respectively;
(16) SEQ ID NOs: 9 and 11, respectively, wherein Xs in position 28, 48, 74 and 79 of SEQ ID NO: 9 are S, I, T and V, respectively, wherein Xs in position of 69, 75 and 84 of SEQ ID NO: 11 are T, V and L, respectively;
(17) SEQ ID NOs: 9 and 11, respectively, wherein Xs in position 28, 48, 74 and 79 of SEQ ID NO: 9 are T, I, T and V, respectively, wherein Xs in position of 69, 75 and 84 of SEQ ID NO: 11 are T, V and L, respectively;
(18) SEQ ID NOs: 7 and 8, respectively, wherein Xs in position 31 and 32 of SEQ ID NO: 8 are A and S, respectively;
(19) SEQ ID NOs: 7 and 8, respectively, wherein Xs in position 31 and 32 of SEQ ID NO: 8 are N and S, respectively;
(20) SEQ ID NOs: 7 and 8, respectively, wherein Xs in position 31 and 32 of SEQ ID NO: 8 are N and A, respectively;
(21) SEQ ID NOs: 7 and 8, respectively, wherein Xs in position 31 and 32 of SEQ ID NO: 8 are N and E, respectively;
(22) SEQ ID NOs: 12 and 13, respectively; or
(23) SEQ ID NOs: 14 and 15, respectively.

6. The isolated monoclonal antibody, or the antigen-binding portion thereof, of claim 1, which is an IgG1, IgG2 or IgG4 isotype.

7. The isolated monoclonal antibody, or an antigen-binding portion thereof, of claim 1, comprising a heavy chain constant region comprising an amino acid sequence of SEQ ID NO: 16, linked to the heavy chain variable region, and a light chain constant region comprising an amino acid sequence of SEQ ID NO: 17, linked to the light chain variable region.

8. The isolated monoclonal antibody, or the antigen-binding portion thereof, of claim 1, which (a) is able to bind human CLDN18.2; (b) is able to bind human CLDN18.1 with low affinity or does not bind human CLDN18.1; (c) is able to be internalized into CLDN18.2⁺ cells, (d) is able to induce antibody-dependent cellular cytotoxicity against CLDN18.2⁺ cells, and/or (e) has anti-tumor activity.

9. The isolated monoclonal antibody, or the antigen-binding portion thereof, of claim 1, which is a mouse, chimeric or humanized antibody or antigen-binding portion thereof.

10. A nucleotide encoding the isolated monoclonal antibody or the antigen-binding portion thereof of claim 1.

11. An expression vector comprising the nucleotide of claim 10.

12. A host cell comprising the expression vector of claim 11.

13. An antibody-drug conjugate, comprising (i) the isolated monoclonal antibody, or antigen-binding portion thereof, of claim 1, and (ii) a toxin.

14. The antibody-drug conjugate of claim 13, wherein the toxin is DT3C or Dxd.

15. A pharmaceutical composition comprising the isolated monoclonal antibody, or antigen-binding portion thereof, of claim 1, and a pharmaceutically acceptable carrier.

16. A pharmaceutical composition comprising the antibody-drug conjugate of claim 13, and a pharmaceutically acceptable carrier.

17. A method for treating a disease associated with high CLDN18.2 expression in a subject in need thereof, comprising administering to the subject a therapeutically effective amount of the pharmaceutical composition of claim 15.

18. The method of claim 17, wherein the disease is tumor or cancer.

19. The method of claim 18, wherein the tumor or cancer is gastric cancer, esophageal cancer, cancer of the gastroesophageal junction, pancreatic cancer, cancer of the bile duct, lung cancer, ovarian cancer, colon cancer, hepatic cancer, head and neck cancer, or gallbladder cancer.

20. A method for treating a disease associated with high CLDN18.2 expression in a subject in need thereof, comprising administering to the subject a therapeutically effective amount of the pharmaceutical composition of claim 16.
